(19) United States
(12) Patent Application Publication
(10) Pub. No.: US 2026/0199559 A1
(43) Pub. Date: Jul. 16, 2026

(54) SYSTEMS AND METHODS FOR FABRICATING HYDROGELS WITH INTRINSIC VASCULAR CHANNELS FOR LIVER TISSUE ENGINEERING

(71) Applicant: 3D BioLabs, LLC, Chadds Ford, PA (US)

(72) Inventors: Joseph Phillip Vacanti, Winchester, MA (US); Tyler Lieberthal, Brookline, MA (US); Craig Neville, Melrose, MA (US); Tatevik Sahakyants, Bedford, NH (US)

(21) Appl. No.: 19/316,780

(22) Filed: Sep. 2, 2025

Related U.S. Application Data
(60) Provisional application No. 63/689,569, filed on Aug. 30, 2024.

(51) Int. Cl. A61L 27/52; A61L 27/24; A61L 27/38; A61L 27/56

(52) U.S. Cl. CPC A61L 27/52; A61L 27/24; A61L 27/38; A61L 27/56; A61L 2400/12

(57) ABSTRACT

Disclosed are implantable devices, systems, and methods of fabricating, testing, optimizing, and/or implanting such devices. An implantable device includes a body, a first intrinsic channel network formed in the body and configured to receive cells, and a second intrinsic channel network formed in the body and configured to allow for active flow of a fluid to support viability and/or function of the cells in the first intrinsic channel network. The first intrinsic channel network comprises a first channel and the second intrinsic channel network comprises a second channel, with at least segments of the first and second channels parallel to and spaced apart from each other. Cells may be suspended in a collagen matrix and introduced to the first intrinsic channel network.

### CROSS-REFERENCE TO RELATED APPLICATION

The present Application claims priority to U.S. Provisional Patent Application No. 63/689,569, entitled “Systems and Methods for Fabricating Hydrogels with Intrinsic Vascular Channels for Liver Tissue Engineering,” filed Aug. 30, 2024, which is hereby incorporated by reference in its entirety for all purposes.

### REFERENCE TO SEQUENCE LISTING SUBMITTED ELECTRONICALLY

The present Application contains a Sequence Listing in XML format submitted electronically herewith via the United States Patent and Trademark Office Patent Center. The contents of the XML copy, created Aug. 29, 2025, is named “119663-5011-US—Sequence Listing” and is 25,591 bytes in size. The Sequence Listing is incorporated herein by reference in its entirety for all purposes.

### BACKGROUND OF THE INVENTION

### Field of the Invention

The present disclosure relates to vascular networks. More particularly, the present disclosure relates to systems and methods for fabricating hydrogels with intrinsic vascular channels for liver tissue engineering.

### Description of Related Art

Orthotopic liver transplantation is an effective intervention for patients grappling with acute or chronic liver failure. Despite successes of this intervention, a severe shortage of donor organs persists, leading to many patients succumbing to their conditions while awaiting transplantation. Even though the number of liver transplants taking place annually in the United States has gradually increased with time, patients remain on the waiting list, and over thousands die annually without receiving a transplant. See A. J. Kwong et al., OPTN/SRTR 2021 Annual Data Report: Liver. Am J Transplant 23, 5178-5263 (2023). To bridge this critical gap, bioartificial liver devices have emerged as temporary hepatic assist machines, potentially offering a lifeline to patients awaiting transplantation or native organ repair and regeneration.

These devices operate by perfusing the patient's plasma through cartridges including a human hepatoma cell line or harvested porcine or human hepatocytes embedded in a scaffold. However, despite numerous clinical trials, the results have generally fallen short of expectations. See He et al, “Bioartificial liver support systems for acute liver failure: A systematic review and meta-analysis of the clinical and preclinical literature,” World J Gastroenterol 25, 3634-3648 (2019).

Limited improvements in clinical benchmarks, such as transient decreases in intracranial pressure and ammonia levels, alongside increases in serum albumin, have failed to demonstrate a statistically significant advantage over simple dialysis. See A. Kanjo et al., Efficacy and safety of liver support devices in acute and hyperacute liver failure: a systematic review and network meta-analysis. Sci Rep 11, 4189 (2021). This limit improvement underscores a need for a paradigm shift in the development of technologies addressing advanced liver disease.

Microfabrication and microfluidic techniques have recently provided a new suite of tools available for tissue engineering applications. For instance, three-dimensional (3D) printing in particular offers the possibility of building de novo tissue architecture with a variety of biologically derived or synthetic polymers. Various 3D printing technologies, including digital light processing (DLP), volumetric printing, and embedded printing have been employed to generate large scaffolds. See B. Grigoryan et al., Multivascular networks and functional intravascular topologies within biocompatible hydrogels. Science 364, 458-464 (2019); P. N. Bernal et al., Volumetric Bioprinting of Complex Living-Tissue Constructs within Seconds. Adv Mater 31, e1904209 (2019); A. Lee et al., 3D bioprinting of collagen to rebuild components of the human heart. Science 365, 482-487 (2019). However, regardless of the printing or manufacturing modality, integration of large tissue engineered scaffolds remains a challenge for such solutions. For instance, a limitation of diffusion to adequately perfuse tissues necessitates embedding vascular channel networks, either by engineering microfluidic channels into the design or neovascular ingrowth to generate the channels.

Thus, prior to the present disclosure there existed a need for forming devices that facilitating scaling of tissue mass for therapeutic tissue replacement applications or the like.

The information disclosed in this Background of the Invention is only for enhancement of understanding of the general background of the invention and should not be taken as an acknowledgement or any form of suggestion that this information forms the prior art already known to a person skilled in the art.

### BRIEF SUMMARY

Given the above background, what is needed in the art are systems and methods for fabricating a device for tissue engineering applications, and such devices.

The present disclosure describes exemplary systems and methods for designing a microchannel network device, fabricating the microchannel network device, seeding a plurality of cells within the microchannel network device, or a combination thereof.

In various embodiments, the present disclosure describes the design, fabrication, and/or evaluation (e.g., by a computer-implemented model) of a channel network device, such as a three-dimensional (3D) printed devices (3DPD), such as for a liver tissue engineering application and/or the like. In some embodiments, the 3DPD includes one or more sets of parallel channels, such as a portal-venous (PV) and a hepatobiliary (HB) channel that are configured for parallel fluid flow within the respective channels, enabling perfusion of a medium, such as whole blood through the PV channel, to induce fluid transfer through the 3DPD device, such as through a nanoporous hydrogel membrane formed at the HB channel. In some embodiments, a plurality of design criteria are utilized to optimize a design of the 3DPD device, such as one or more fluid flow design criteria, one or more heat transfer design criteria, one or more electromagnetic criteria, one or more chemical reaction criteria, one or more structural design criteria, or a combination thereof. In some embodiments, a method for fabricating the 3DPD includes utilizing a 3D printing apparatus with a pre-polymer solution, such as Gelatin methacryloyl (GelMA) PEGDA and a photoink, followed by cell seeding by flowing a collagen hydrogel suspension through the HB channel. However, the present disclosure is not limited thereto.

In some embodiments, perfusion of rhodamine through the PV channel provides enhanced mass transfer into the bulk of the hydrogel compared to non-perfused devices and significantly elevated concentrations in the HB. Furthermore, in some embodiments, perfusion of medium provides enhanced viability and function of cells, as evidenced by the present invention. In some embodiments, viability assays and gene expression analysis revealed maintenance of cell viability and proliferation over time within the implantable devices of the present disclosure, along with improved liver-specific functions. In some embodiments, similar results were observed with primary rat hepatocytes. In some embodiments, co-culture experiments involving primary rat hepatocytes, endothelial cells, and mesenchymal stem cells in 3DPDs showed enhanced viability, broad liver-specific gene expression, and histological features indicative of liver tissue architecture. In some embodiments, in vivo implantation of 3DPDs in a rat renal shunt model demonstrated successful blood flow through the devices without clot formation and maintenance of cell viability.

More particularly, in some embodiments, the present disclosure describes designing an implantable liver device and/or creating a bioreactor environment mimicking hepatic physiology enhancing cell viability and function within the implantable liver device. In some embodiments, the present disclosure combines additive manufacture techniques such as 3D printing microfabrication, cell biology, and surgical techniques into a design workflow to design, fabricate, and/or utilize a novel, highly scalable bi-channel liver assist device configured for direct implantation within a subject, with circulatory anastomosis in vivo is provided. In some embodiments, flow if a medium such as blood through the implantable device of the present disclosure generates mass transport across a nanoporous barrier of the implantable device, such as in order to nourish parenchymal tissue in parallel channels. In some embodiments, the workflow includes evaluating one or more device designs in vitro to optimize design choices prior to validation in vivo (e.g., in a renal shunt model).

The systems, methods, and devices of the present invention have other features and advantages that will be apparent from, or are set forth in more detail in, the accompanying drawings, which are incorporated herein, and the following Detailed Description, which together serve to explain certain principles of exemplary embodiments of the present invention.

Other features and advantages of the invention will be apparent from, or are set forth in more detail in, the accompanying drawings, which are incorporated herein, and the following Detailed Description, which together serve to explain certain principles of exemplary embodiments of the present invention.

### BRIEF DESCRIPTION OF THE DRAWINGS

FIG. 1 illustrates a block diagram illustrating an embodiment of a system for fabricating a microchannel network device and/or seeding a plurality of cells in a microchannel device, in accordance with embodiments of the present disclosure.

FIG. 2 illustrates a fabrication system for fabricating a microchannel network device, in accordance with embodiments of the present disclosure.

FIG. 3 illustrates a bioreactor system for controlling flow through a microchannel network device, in accordance with embodiments of the present disclosure.

FIG. 4 illustrates a client device for designing and/or controlling a flow through a microchannel network device, in accordance with embodiments of the present disclosure.

FIGS. 5A through 5F collectively illustrate design, fabrication, and evaluation of 3D printed devices (3DPDs), in accordance with some exemplary embodiments of the present disclosure. (FIG. 5A) The general design motif of the 3D printed channels consisted of two parallel channels comprising a hepatobiliary (HB) channel and portal-venous (PV) channel. Perfusion of the PV channel by blood or medium induces water and small molecule transfer through the nanoporous hydrogel barrier to drain into the HB. (FIG. 5B) The workflow to produce 3DPDs included 3D printing from a CAD file with a PEGDA photoink on a LUMEN-X DLP printer. Printed devices were then seeded with cells through HB channels in a suspension of collagen hydrogel. (FIG. 5C) Finished 3DPDs showing PV channel (red) and HB channel (blue). SEM and brightfield images show channels separated by three printed layers. (FIG. 5D) Red dye perfused through the PV channel led to enhanced mass transfer into the bulk of the hydrogel in comparison to non-perfused devices. (FIG. 5E) Rhodamine perfusion through the PV was detected in the HB at 30 min and 60 min. Concentrations in the HB were significantly elevated with perfusion. (FIG. 5F) Increasing flow rates in the PV led to nL/s flow rates in the HB when the HB outlet was open. Data represent means±SD for N=3-6 devices.

FIGS. 6A through 6G collectively illustrate perfusion in 3DPDs enhances viability and function of H4-II-E-C3 rat hepatoma cells, in accordance with embodiments of the present disclosure. (FIG. 6A) Fluorescence, brightfield, and hematoxylin and eosin (H&E) staining of cell/collagen hydrogels within 3DPDs. After culture in bioreactors, cell/collagen constructs were removed and stained with propidium iodide. Centers of cell death within the center of the collagen hydrogel were observed without perfusion. (FIG. 6B) High densities of cells (20 million/mL) were loaded into 3DPDs. ATP was quantified in total 3DPD lysates as a measure of cell viability using CellTiter Glo 3D. Data are plotted as fold-changes of arbitrary luminescence units. Perfusion maintained viable cells at 24 hours and permitted cell proliferation at 3 and 7 days. (FIG. 6C) Brightfield and H&E imaging of rat hepatoma cells at 1, 3, and 7 days in 3DPDs with perfusion. (FIG. 6D) Relative gene expression of proliferation (PCNA) and apoptosis (ratio of Bax/Bcl2) between flow and static conditions at 24 hours. (FIG. 6E) Caspase 3/7 enzyme activity was determined in total 3DPD lysates as a measure of apoptosis. (FIG. 6F) Quantification of urea and albumin concentration in medium supernatant and within hydrogel. (FIG. 6G) Comparison of gene expression by qPCR of cells cultured in perfused 3DPDs or on tissue culture polystyrene (TCPS). (FIG. 6H) qPCR gene expression between perfused and static 3DPDs. Data represent means±SD for N=3-11 devices.

FIGS. 7A-7H collectively illustrate perfusion in 3DPDs enhances viability and function of primary rat hepatocytes, in accordance with embodiments of the present disclosure. (FIGS. 7A-7B) Isolated rat hepatocytes suspended in collagen hydrogels at concentrations between 1 and 8 million/mL and loaded into 3DPDs. At 3 and 7 days, viability and albumin production were determined. Viability data are plotted as fold-changes of arbitrary luminescence units. (FIG. 7C) Viability and albumin production of 4 million/mL cryopreserved hepatocytes determined. (FIG. 7D) Relative gene expression of p450 genes (CYP1A1, CYP1A2, and CYP3A1) and G6PC in perfused or static 3DPDs. (FIG. 7E) 3DPDs were cultured for 3 days, after which cell/collagen gels were removed from HB channels and treated with a CYP3A4 substrate. Enzyme activity was determined with p450-Gbo assay. (FIG. 7F) Perfused 3DPDs were treated with vehicle or rifampicin for 24 hours. CYP3A4 activity was then determined using the p450-Gbo assay. (FIG. 7G) Experimental setup to determine relative contribution of bulk diffusion through the 3DPD hydrogel. In the default flow setup (left), a peristaltic pump fed medium into the PV channel, after which it drained into the bioreactor chamber. The pump then recirculated medium from the chamber back into the PV. In an alternative setup (right), external medium was in a separate chamber and only entered the 3DPD via the PV channel. Analogous static setups were constructed by submerging devices in medium (static, media outside) or not (static, no media outside). (FIG. 7H) Viability of cells in the HB was determined after 3 days of culture in each setup by CellTiter Glo 3D and representative fluorescent microscopy images of cell/collagen hydrogels in 3DPDs treated with calcein AM (green), propidium iodide (red), and Hoechst (blue). Data represent means±SD for N=3-8 devices.

FIGS. 8A-8D collectively illustrate perfusion of 3DPDs including co-cultures of primary rat hepatocytes, endothelial cells (ECs), and mesenchymal stem cells (MSCs) enhances viability and broad liver-specific gene expression in accordance with embodiments of the present disclosure. (FIG. 8A) Viability and albumin production of mixed cultures at day 3 and 7. Viability data are plotted as fold-changes of arbitrary luminescence units. (FIG. 8B) Representative histology of co-cultures in 3DPDs with flow. (Left) H&E staining, (middle) fluorescence imaging of ECs in red and nuclei in cyan. Circled area indicates hepatocyte rosette structure, (right) fluorescence imaging of MRP2 bile transporter in purple and nuclei in cyan. (FIG. 8C) RNA-seq was performed on 3DPDs cultured for 7 days. Normalized expression differences were evaluated against the hallmark and C2 collections of the Human Molecular Signatures Database. (FIG. 8D) Select genes from RNA-seq dataset representing liver synthetic products, blood clotting factors, bile synthesis and transport, and p450s. Data represent means±SD for N=3-9 devices.

FIGS. 9A through 9E collectively illustrate in vivo implantation of 3DPDs in a rat renal shunt model, in accordance with embodiments of the present disclosure. (FIG. 9A) Schematic of channel design for in vivo implantation. Silastic tubing was used to cannulate the renal artery and vein for inflow and outflow. A separate port was added to allow for continuous heparin delivery into the PV channel via an osmotic pump. (FIG. 9B) Representative images of implanted devices after establishing blood flow intraoperatively and when explanting at post-op day 2. Bright red blood indicates that devices were free of large clots that occluded flow. (FIG. 9C) At post-op days 1 and 2, devices were removed from the abdomen and the outflow tubing was severed. The flow rate through the device was recorded by collecting blood over time. (FIG. 9D) Rat hepatoma cells (20 million/mL) were loaded in 3DPDs and, after letting the collagen set, immediately implanted. A second cell-seeded device was placed in the abdomen as a non-perfused control. Devices were recovered at 1 and 2 days to be analyzed by Cell Titer Glo 3D. Viability data are plotted as fold-changes of arbitrary luminescence units. (FIG. 9E) Rat hepatocytes were isolated and loaded into 3DPDs at 4 million hepatocytes/mL. Devices were cultured in bioreactors for 3 days and then implanted in a littermate intravascularly (“flow”) or abdominally (“static”) for 1 day prior to analysis. A second group of 3DPDs (“in vitro”) were not implanted and continued culture in bioreactors for a fourth day. Data represent means±SD for N=3-6 animals or devices

FIGS. 10A and 10B collectively illustrate port optimization and burst pressure testing, in accordance with embodiments of the present disclosure. (FIG. 10A) Port-tube connections were optimized for fit through COMSOL mechanical simulations of the hydrogel and CAD design. (FIG. 10B) 3DPDs were injected with PBS via syringe pump with the outlet port closed. A pressure sensor was introduced to the heparin port to measure burst pressures. The site of failure was recorded as a tube-port disconnection (at the PV inlet, PV outlet, or heparin port) or a PV-HB membrane failure.

FIG. 11 illustrates an in vitro bioreactor setup including 3D printed device, housing, silastic tubing, and peristaltic pump, in accordance with embodiments of the present disclosure.

FIGS. 12A-12C collectively illustrate hepatocyte performance in different biologically derived materials, in accordance with embodiments of the present disclosure. (FIG. 12A) Co-cultures of hepatocytes, endothelial cells, and mesenchymal stem cells were generated in 3D gels of collagen, fibrin, Matrigel, or mixtures of these in glass bottom dishes. Supernatants were collected and assayed for albumin concentration using an ELISA. (FIG. 12B) Gels were imaged and the diameter measured to determine percent contraction relative to the diameter at setup. (FIG. 12C) 3DPDs were cultured for 7 days with a co-culture of hepatocytes, endothelial cells, and mesenchymal stem cells. Cell/collagen constructs were removed from 3DPDs and immunostained whole with HNF4α and Ki67. Gels were cleared using FocusClear and imaged on a fluorescent microscope. Arrow indicates a HNF4α+/Ki67+ proliferating hepatocyte.

FIG. 13 illustrates in vitro culture of 3DPDs with H4-II-E-C3 rat hepatoma cells with or without bulk media diffusion, in accordance with embodiments of the present disclosure. Bioreactors were set up according to diagram in FIG. 7G. Total viability of cells in 3DPD was determined by CellTiter Glo 3D.

FIGS. 14A-14I illustrate scaling of two-channel design with fractal geometry, in accordance with some exemplary embodiments of the present disclosure. (FIG. 14A-14C) CAD images of scaled design with PV channels in grey and HB channels in blue. (FIG. 14D-FIG. 14F) 3D printed devices with channels having a diameter that is micron to millimeter size filled with polyurethane for visualization. PV channels are in white and HB channels in blue. (FIG. 14G) Scaled 3DPDs were loaded with rat hepatoma cells and viability was determined by CellTiter Glo 3D at indicated time points. Data are plotted as fold-changes of arbitrary luminescence units. (FIG. 14H) Representative propidium iodide (PI) dead cell staining of cells in HB channels at day 1. (FIG. 14I) Intraoperative implantation of scaled 3DPD in a rodent renal shunt model. Data represent means±SD for N=3-4 devices.

FIGS. 15A-15C illustrate PEGDA hydrogel swelling properties, in accordance with some exemplary embodiments of the present disclosure. (FIG. 15A) Summary of hydrogel properties using the mass in the dry state (m_{d}) or swollen state (mₛ) including porosity in water (1−n_d/m_s)*100, swelling ratio in PBS

$$\frac{{m}_{s} - {m}_{d}}{{m}_{d}} ,$$

and sol fraction of pre-polymer that elutes after polymerization

$$1 - \frac{{m}_{d ( post - swell )}}{{m}_{d ( post - print )}} .$$

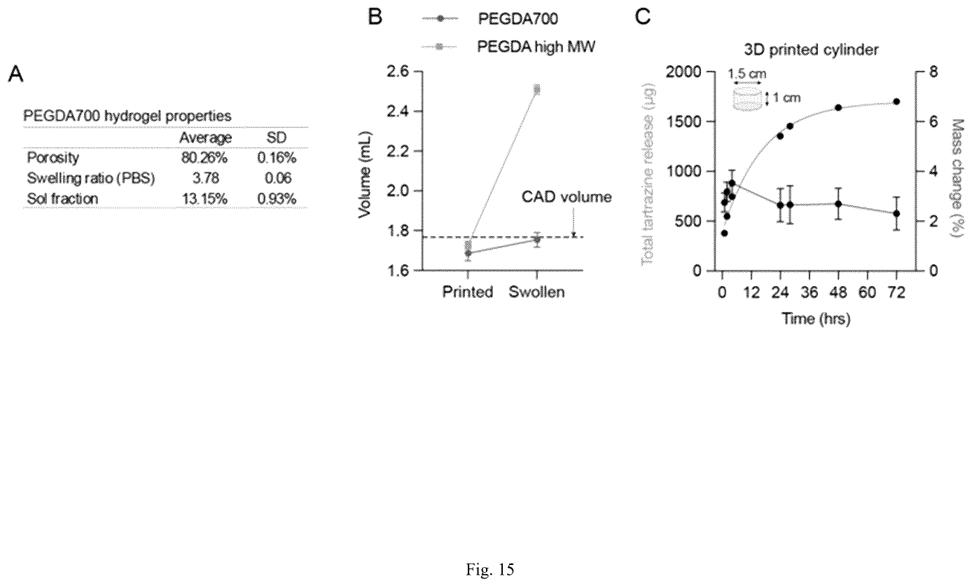

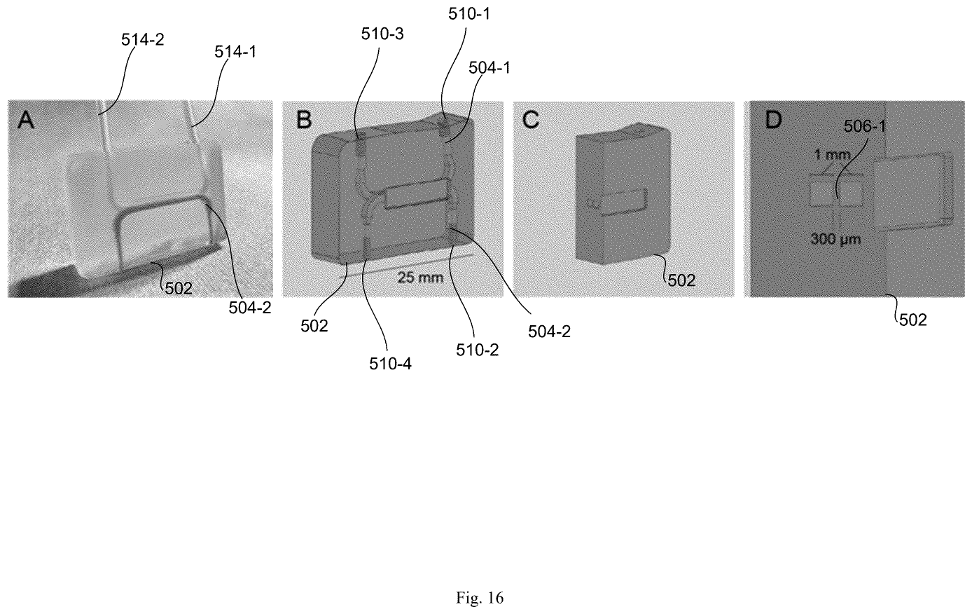

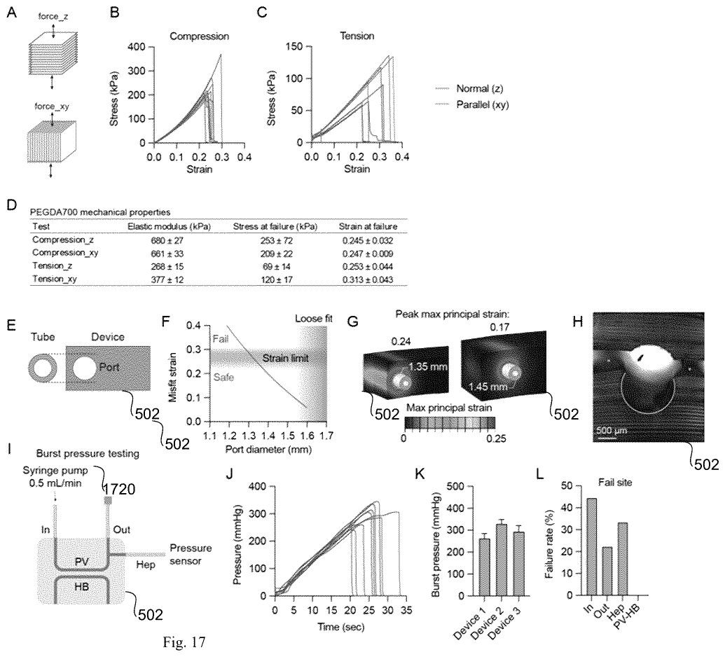

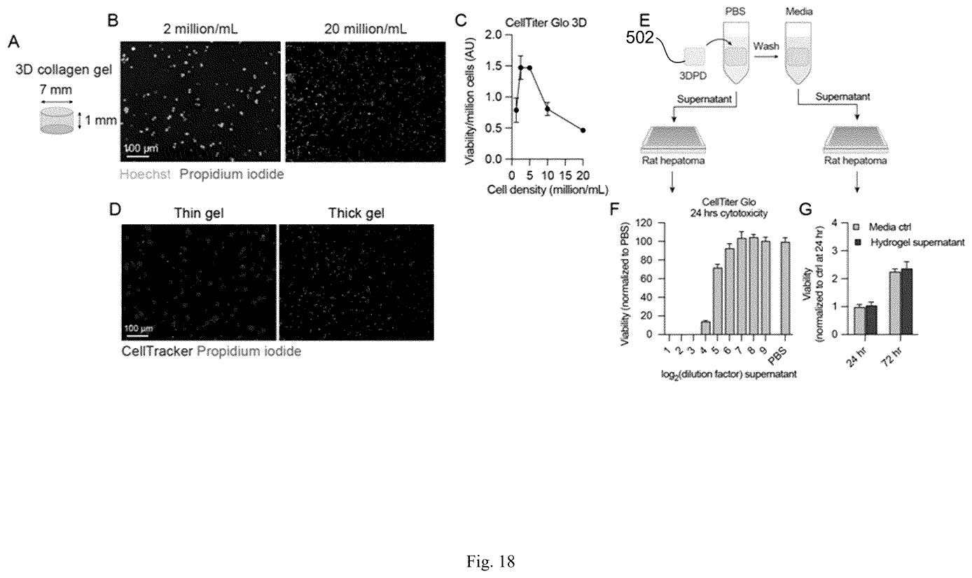

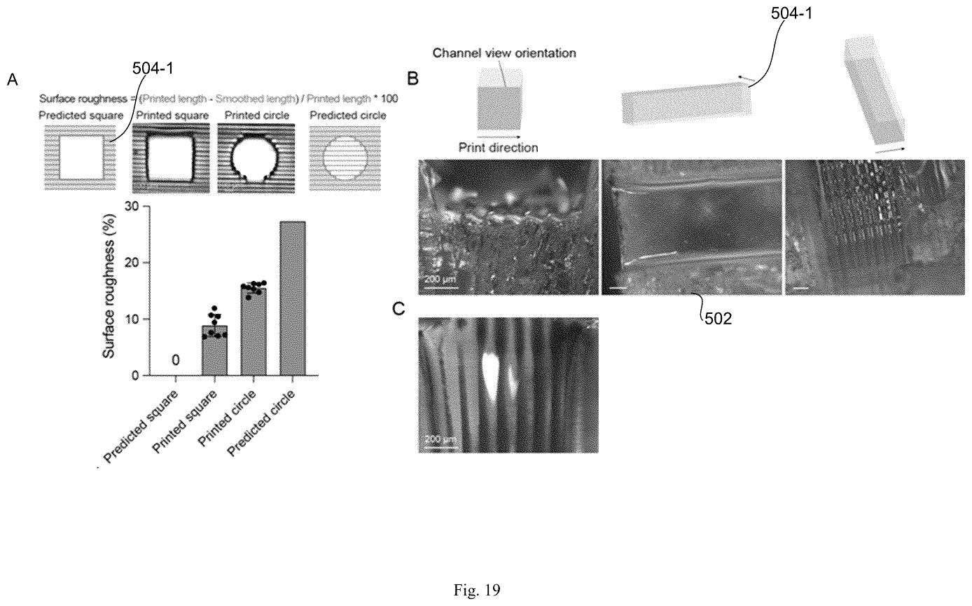

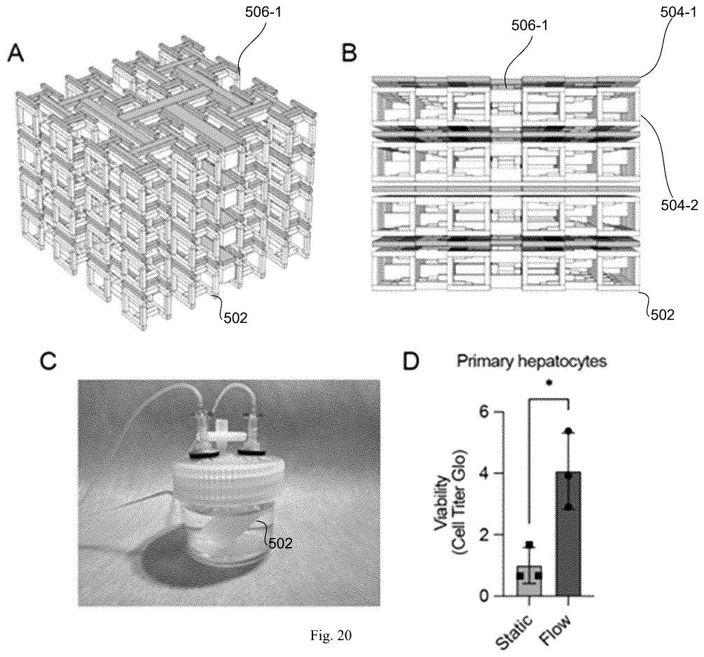

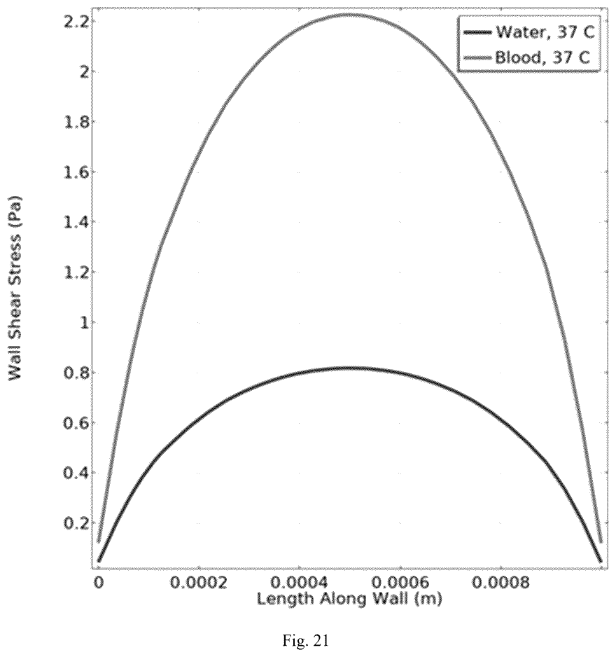

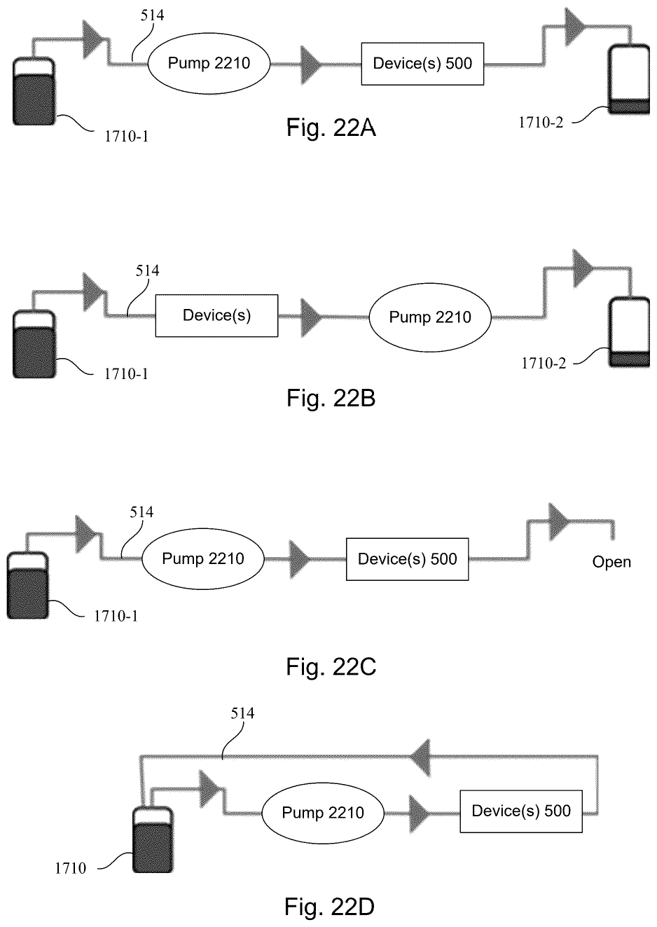

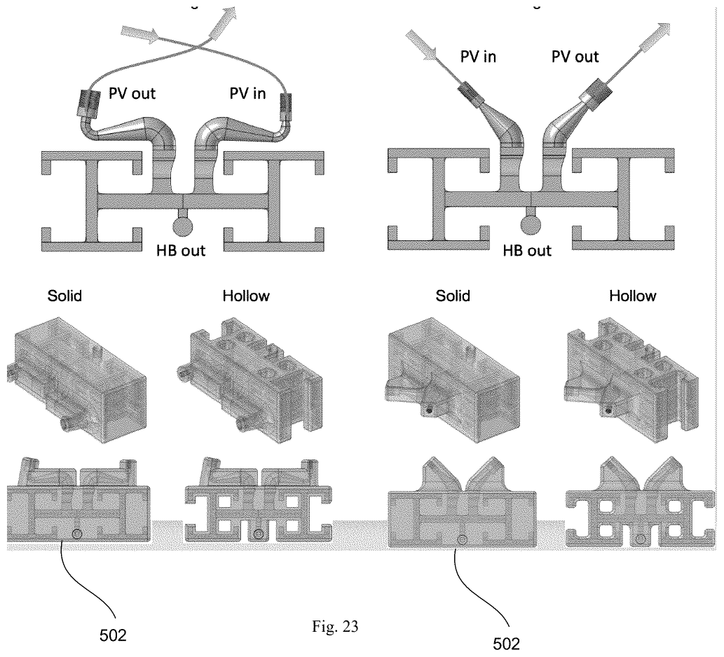

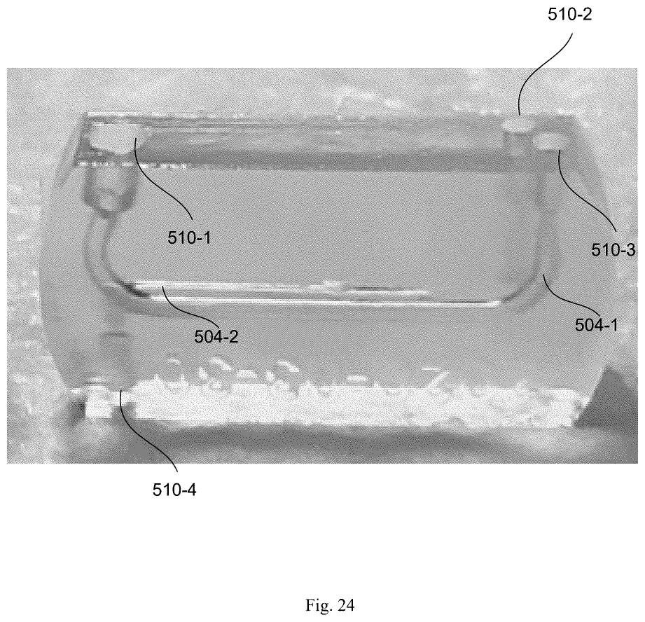

(FIG. 15B) Representative cylinders were printed in PEGDA and (post-print) (MW 700 Da) or a high molecular weight commercial ink and swollen in PBS. The volume of the gels after printing and after swelling were recorded. Dotted line shows the theoretical volume from the CAD dimensions. (FIG. 15C) PEGDA700 cylinders were washed out in PBS over 72 hrs. Cumulative release of the photomask (tartrazine) was determined by measuring the absorbance of supernatants at 430 nm (orange—left y-axis). The mass of gels was also recorded (green—right y-axis).

FIGS. 16A-16D illustrate a 3D printed device, in accordance with some exemplary embodiments of the present disclosure. (FIG. 16A) 3D printed device with channels filled with polyurethane for visualization. PV channel in white and HB channel in blue. (FIG. 16B-16D) Additional CAD views of a two-channel device.

FIGS. 17A-17L illustrate mechanical testing of 3D printed 20% PEGDA (MW 700 Da), port optimization, and burst pressure testing, in accordance with some exemplary embodiments of the present disclosure. (FIG. 17A) Mechanical testing of hydrogel samples was conducted on vertically printed layers (z) in which force is applied normal to layers or on horizontally printed samples (xy) in which force is applied parallel to layers. (FIG. 17B) Stress-strain curves of PEGDA in compression. (FIG. 17C) Stress-strain curves of PEGDA in tension. (FIG. 17D) Summary of mechanical properties of PEGDA. (FIG. 17E) Port diameters are slightly smaller than tube outer diameters to ensure tight fit without cracking of the hydrogel. (FIG. 17F) To help determine the optimal port diameter, a misfit strain metric

$${ε}_{port} ∼ \frac{\text{?} ( D {\text{?}}_{tube} - {D}_{port} )}{{D}_{port}} * \frac{{E}_{tube}}{{E}_{port} + {E}_{tube}}$$

with diameters D and elastic modulus E is plotted vs. port diameter along with observed levels of tubing fit, and compared to the strain at failure listed in FIG. 17D. (FIG. 17G) Location and design of port-tube connections were further optimized for fit and to reduce strain concentration in the hydrogel by Abaqus solid mechanics simulations. Shown are a device with a 1.35 mm diameter port (left) and a larger 1.45 mm diameter port with additional wall thickness (right). The smaller 1.35 mm diameter port is likely to crack at the site of tube insertion based on predicted misfit strain and measured ultimate strain at failure. (FIG. 17H) Testing of a 3D printed 1.35 mm diameter port when tubing is inserted. Outlines show port boundaries. Asterisks indicate split layers along the interface after attempting to insert tubing. (FIG. 17I-FIG. 17K) 3DPDs were injected with PBS via syringe pump with the outlet port closed. A pressure sensor was introduced to the heparin port to measure burst pressures. (FIG. 17L) The site of failure was recorded as a tube-port disconnection (at the PV inlet, PV outlet, or heparin port) or a PV-HB barrier failure.

FIGS. 18A-18G illustrate rat hepatoma cell density selection and PEGDA biocompatibility, in accordance with some exemplary embodiments of the present disclosure. (FIG. 18A) 3D collagen gels were formed in glass bottom dishes for a range of cell densities. The thickness of the gel was 1 mm. (FIG. 18B) Propidium iodide dead cell staining of low and high density 3D cultures. (FIG. 18C) Viability of whole 3D collagen gels was determined with CellTiter Glo 3D and signals were normalized to total cell number in each gel. Too-low densities (1 million/mL) and too-high densities (20 million/mL) were suboptimal. (FIG. 18D) Dead cell staining of high cell density cultures in thin or thick gels. (FIG. 18E) Biocompatibility of the PEGDA hydrogel was determined by culturing cells in the wash buffer of the hydrogels following printing. (FIG. 18F) Several washes were required to remove leachable pre-polymer components as initial washes of the hydrogel were acutely cytotoxic. (FIG. 18G) After washing, hydrogel wash buffer supernatants did not impact viability or proliferation.

FIGS. 19A-19C illustrate effects of channel cross-sectional geometry, in accordance with some exemplary embodiments of the present disclosure. (FIG. 19A) Due to layer-by-layer printing, circular cross-sections result in a “staircase” type surface topology whereas rectangular cross-sections have two faces that are composed of monolithic layers, producing a smoother surface. (FIG. 19B) Stereoscopic images of channel surface features with reference orientations (above). (FIG. 19C) A tendency for blood cells to accumulate in the valleys between printed layers was observed in explanted 3DPDs.

FIGS. 20A-20D illustrate scaling of two-channel design with fractal geometry, in accordance with some exemplary embodiments of the present disclosure. (FIG. 20A-20B) Channel volumes can be increased infinitely by tuning branching (fractal generations) and stacking of layers. Shown is an example of three H-branches or six generations. (FIG. 20C) Larger bioreactor housing was developed using 60 mL wide mouth jars and the previous peristaltic perfusion system. (FIG. 20D) Viability of primary hepatocytes in scaled 3DPDs at day 3. Data represent means±SD for N=3 devices.

FIG. 21 illustrates COMSOL simulations using either water or blood fluid properties, in accordance with some exemplary embodiments of the present disclosure. The simulations were run with 5 mL/min fluid flow. The resulting wall shear stress (Pa) is plotted against the wall position (corner-to-corner).

FIGS. 22A, 22B, 22C, and 22D collectively illustrate various bioreactor systems including one or more devices and one or more pumps.

FIG. 23 illustrates a first implantable device, in accordance with embodiments of the present disclosure.

FIG. 24 illustrates a first implantable device, in accordance with embodiments of the present disclosure

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the Figures, reference numbers refer to the same or equivalent parts of the present invention throughout the several Figures of the drawing.

### DETAILED DESCRIPTION

Blood perfusion is a significant requirement for tissue viability that greatly limits scalability of potential functional tissue engineered organ implants. The present disclosure provides a 3D printed hydrogel device containing parallel channels having a diameter that is micron to millimeter size where blood flow supports functional liver tissue through nanoporous material. In vitro, medium actively pumped through the device enhanced viability and function of hepatocytes. After subsequent rounds of design optimization, evaluation of the device in a renal rodent shunt model demonstrated that blood perfusion through the channel significantly enhanced cell viability and function relative to non-perfused implants. The intrinsic architecture of the device facilitates scaling of tissue mass, thus holding promise for therapeutic tissue replacement applications.

Reference will now be made in detail to various embodiments of the present invention(s), examples of which are illustrated in the accompanying drawings and described below. While the invention(s) will be described in conjunction with exemplary embodiments, it will be understood that the present description is not intended to limit the invention(s) to those exemplary embodiments. On the contrary, the invention(s) provides not only the exemplary embodiments, but also various alternatives, modifications, equivalents and other embodiments, which may be included within the spirit and scope of the invention as defined by the appended claims.

In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first graphical chart could be termed a second graphical chart, and, similarly, a second graphical chart could be termed a first graphical chart, without departing from the scope of the present disclosure. The first graphical chart and the second graphical chart are both graphical charts, but they are not the same graphical chart.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The present description includes example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The present invention, for purpose of explanation, is described with reference to specific implementations. The illustrative discussions below, however, are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if (a stated condition or event) is detected” may be construed to mean “upon determining” or “in response to determining” or “upon detecting (the stated condition or event)” or “in response to detecting (the stated condition or event),” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, +10%, +5%, or 1% of a given value.

Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to +5%.

As used herein, the term “equally spaced” means that a distance from a first feature to a corresponding second feature is the same for successive pairs of features unless expressly stated otherwise.

“Biodegradable,” as used herein, means materials that are bioresorbable and/or degrade and/or break down by mechanical degradation (e.g., dissolve, resorb, etc.) upon interaction with a physiological environment into components that are metabolizable or excretable, over a period of time from minutes to three years, preferably less than one year, while maintaining the requisite structural integrity.

“Exchange mechanism,” as used herein, means a material or structure configured to substantially allow or inhibit a flow of material from a first element to a second element including fenestrated walls, permeable membranes, permeable walls, porous walls, porous membranes, perforations, and the like.

“Diameter,” as used herein, is inclusive of equivalent characteristic lengths including hydraulic diameters of non-circular structures.

“Flush,” as used herein, means a surface of a first element and a coplanar surface of a second element to have a distance, or level, separating the first element and the second element to be within a tolerance of about 0 μm, within a tolerance of about 5 m, within a tolerance of about 10 μm, within a tolerance of about 20 μm, or within a tolerance of about 100 μm.

“Direct flow,” as used herein, means a transfer or a flow of at least one substance or material from a first element to at least a second element.

“Indirect flow,” as used herein, means an exchange or flow of at least one substance or material from a first element to at least a second element which is mediated by an exchange mechanism.

“Natural manner,” as used herein, means a process or development as found in nature.

“Polymer,” as used herein, includes polymers, and monomers that can be polymerized or adhered to form an integral unit. The polymer can be non-biodegradable or biodegradable, e.g., via hydrolysis or enzymatic cleavage.

“Subsequent channel,” as used herein, means, for a given channel, a channel which material flows therefrom. Accordingly, by “preceding channel,” as used herein, is meant, for the given channel, a channel which material flows thereto.

“Rigid,” as used herein, means a material that is stiff and does not deform easily. By “elastomeric,” as used herein, is meant a material or a composite material that is not rigid as defined herein.

Additionally, the terms “client,” “patient,” “subject,” and “user” are used interchangeably herein unless expressly stated otherwise.

A “disease” is a state of health of an animal wherein the animal cannot maintain homeostasis, and wherein if the disease is not ameliorated then the animal's health continues to deteriorate. In an exemplary embodiment, the device of the invention is implanted into a subject afflicted with a disease and in treatment thereof by means of such implantation.

“Amino Acid,” as used herein refers to the genus encompassing hydrophilic amino acids, acidic amino acids, basic amino acids, polar amino acids, hydrophobic amino acids, aromatic amino acids, non-polar amino acids and aliphatic amino acids, including the genus and the species therein. A “peptide” is formed from such amino acids linked via peptide bonds. Amino acids also encompass amino-carboxylic acid species other than α-amino acids, e.g., aminobutyric acid (aba), aminohexanoic acid (aha), aminomethylbenzoic acid (amb) etc. In an exemplary embodiment, the cyanine dye of the invention is conjugated to a carrier molecule through a linker having one or more than one amino acid. Exemplary amino acids of use in such linkers include lysine, proline and acidic amino acids.

“Activated derivatives of carboxyl moieties,” and equivalent species, refers to moiety on a precursor component of a conjugate of the invention (e.g., dye, adaptor, linker, polyvalent moiety) having a leaving group, e.g., an active ester, acyl halide, acyl imidazole, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include mono-, di- and multivalent radicals, having the number of carbon atoms designated (i.e., C₁-C₁₀ means one to ten carbons). Examples of saturated alkyl radicals include, but are not limited to, groups such as methyl, methylene, ethyl, ethylene, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, optionally, those derivatives of alkyl defined in more detail below, such as “alkenyl”, “alkynyl”, “alkyldiyl”, “alkyleno” and “heteroalkyl.”

“Alkenyl” refers to an unsaturated branched, straight-chain or cyclic alkyl radical having at least one carbon-carbon double bond derived by the removal of one hydrogen atom from a single carbon atom of a parent alkane. The radical may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc., and the like. In exemplary embodiments, the alkenyl group is (C₂-C₆) alkenyl.

“Alkynyl” refers to an unsaturated branched, straight-chain or cyclic alkyl radical having at least one carbon-carbon triple bond derived by the removal of one hydrogen atom from a single carbon atom of a parent alkene. Typical alkynyl groups include, but are not limited to, ethynyl; propynyls such as prop-1-yn-1-yl, prop-2-yn-1-yl, etc.; butynyls such as but-1-yn-1-yl, but-3-yn-1-yl, etc., and the like. In exemplary embodiments, the alkynyl group is (C₂-C₆) alkynyl.

“Alkyldiyl” refers to a saturated or unsaturated, branched, straight-chain or cyclic divalent hydrocarbon radical derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyls include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,3-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. In preferred embodiments, the alkyldiyl group is (C₂-C₆) alkyldiyl. Also preferred are saturated acyclic alkanyldiyl radicals in which the radical centers are at the terminal carbons, e.g., methandiyl (methano); ethan-1,2-diyl(ethano); propan-1,3-diyl(propano); butan-1,4-diyl(butano), and the like (also referred to as alkylenos, defined infra).

“Alkyleno” refers to a straight-chain alkyldiyl radical having two terminal monovalent radical centers derived by the removal of one hydrogen atom from each of the two terminal carbon atoms of straight-chain parent alkane, alkene or alkyne. Typical alkyleno groups include, but are not limited to, methano; ethylenos such as ethano, etheno, ethyno; propylenos such as propano, prop(1)eno, propa(1,2)dieno, prop(1)yno, etc.; butylenos such as butano, but(1)eno, but(2)eno, buta(1,3)dieno, but(1)yno, but(2)yno, but(1,3)diyno, etc., and the like. Where specific levels of saturation are intended, the nomenclature alkano, alkeno and/or alkyno is used. In preferred embodiments, the alkyleno group is (C₂-C₆) alkyleno.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, P and S, and wherein the nitrogen and sulfur atoms may optionally be oxidized and the nitrogen heteroatom may optionally be quaternized. The heteroatom(s) O, N, S, P and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,-S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃.

Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl,” by themselves or in combination with other terms, represent, unless otherwise stated, cyclic versions of “alkyl” and “heteroalkyl,” respectively. Also included are di- and multi-valent species such as “cycloalkylene.” Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C1-C₄)alkyl” is meant to include, but not be limited to, species such as trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that include from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Also included are di- and multi-valent linker species, such as “arylene.” Substituents for each of the above noted aryl and heteroaryl ring systems are selected from the group of acceptable substituents described below.

For brevity, the term “aryl”, when used in combination with other terms (e.g., aryloxy, arylthioxy, arylalkyl) includes aryl and, optionally, heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl,” and “heteroaryl”) include both substituted and unsubstituted forms of the indicated radical. Exemplary substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, =NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, SO₃R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. Accordingly, from the above discussion of substituents, one of skill in the art will understand that the terms “substituted alkyl” and “heteroalkyl” are meant to include groups that have carbon atoms bound to groups other than hydrogen atoms, such as haloalkyl (e.g., —CF₃ and —CH₂CF₃) and acyl (e.g., —C(O)CH₃, —C(O)CF₃, —C(O)CH₂OCH₃, and the like).

The substituents set forth in the paragraph above are referred to herein as “alkyl group substituents.”

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′,—S(O)R′, —S(O)₂R′, SO₃R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula —T—C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula -(CRR′)s-X-(CR″R′)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

The substituents set forth in the two paragraphs above are referred to herein as “aryl group substituents.”

A “linkage fragment” is a bond, or is a group that is formed by reaction of two reactive functional groups of complementary reactivity. An exemplary linkage fragment is an amide formed by the reaction of an amine and an activated derivative of a carboxylic acid (e.g., acyl halide, acyl imidazole, active ester, etc.). When the cyanine dyes of the invention are conjugated to a carrier molecule, they can be conjugated directly through a linkage fragment or through a linker that includes one or more linkage fragment. For example, a conjugate in which the dye is bound to a carrier molecule through a linker optionally includes a linkage fragment joining the linker and the dye and/or joining the linker and the carrier molecule.

The term “Linker” or “L,” as used herein, refers to a single covalent bond or a series of stable covalent bonds incorporating 1-40, e.g., 10-30 nonhydrogen atoms selected from the group consisting of C, N, O, S and P that covalently attach the fluorogenic or fluorescent compounds to another moiety such as a chemically reactive group or a biological or non-biological component, e.g., a carrier molecule. Exemplary linkers include one or more linkage fragment, e.g., —C(O)NH—, —C(O)O—, —NH—, —S—, —O—, joining the dye to the linker and/or the linker to the carrier molecule and the like. Linkers are also of use to join the cyanine nucleus component of the compound to a reactive functional group, or a component of a reactive functional group.

As used herein, “fluorophore” refers to a fluorescent species.

“Moiety” refers to the radical of a molecule that is attached to another moiety.

Furthermore, when a reference number is given an “i^{th}” denotation, the reference number refers to a generic component, set, or embodiment. For instance, a subject termed “subject i” refers to the i^{th} subject in a plurality of subject (e.g., a subject 1-i in a plurality of subjects 1).

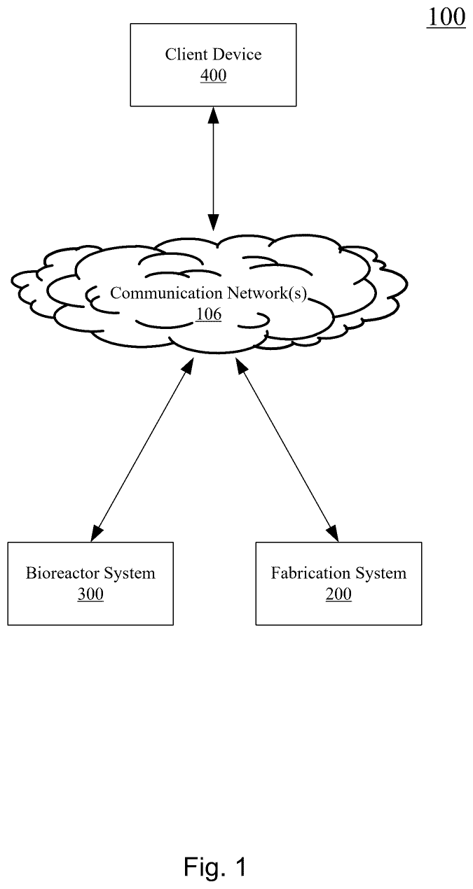

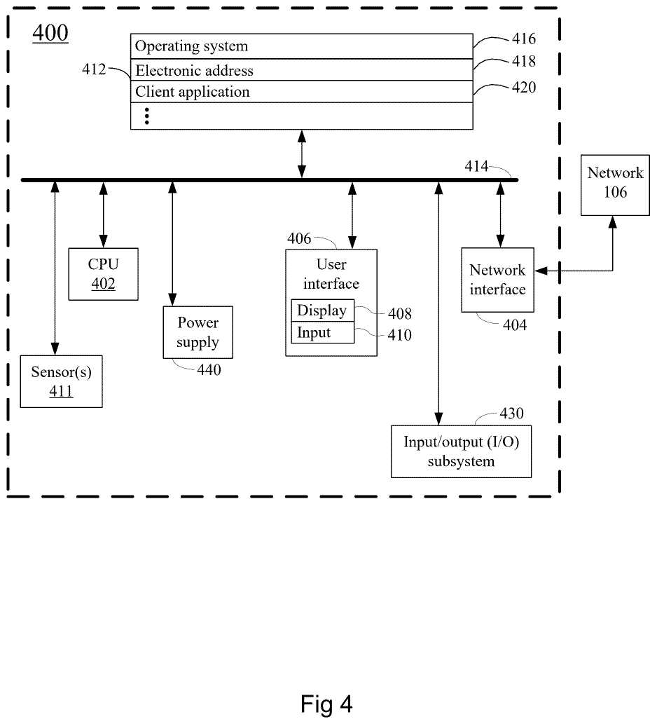

In the present disclosure, unless expressly stated otherwise, descriptions of devices and systems will include implementations of one or more computers. For instance, and for purposes of illustration in FIG. 1, a client device (e.g., client device 400 of FIG. 4) is represented as single device that includes all the functionality of the client device 400. However, the present disclosure is not limited thereto. For instance, the functionality of the client device 400 may be spread across any number of networked computers and/or reside on each of several networked computers and/or by hosted on one or more virtual machines and/or containers at a remote location accessible across a communications network (e.g., communications network 106). One of skill in the art will appreciate that a wide array of different computer topologies is possible for the client device 400, and other devices and systems of the preset disclosure, and that all such topologies are within the scope of the present disclosure.

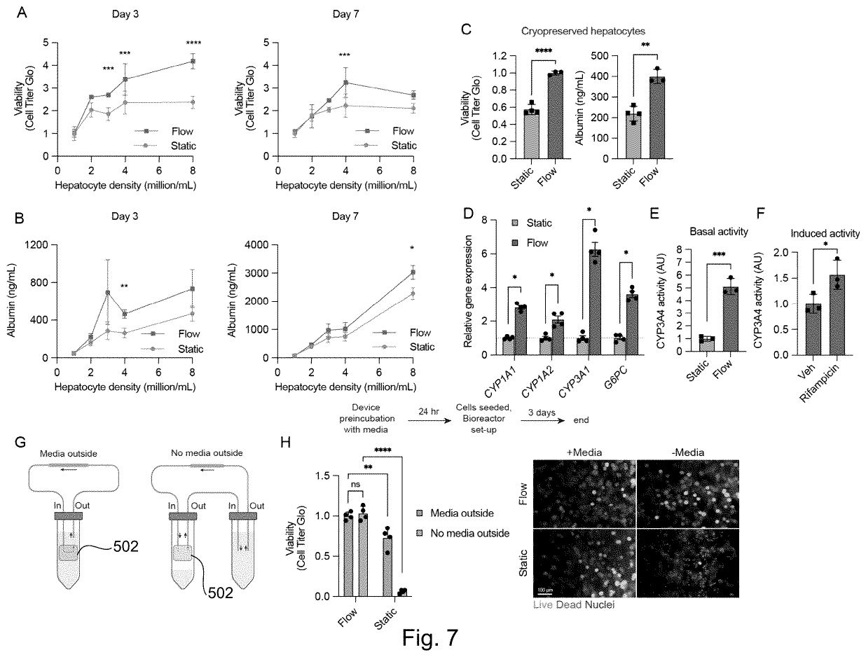

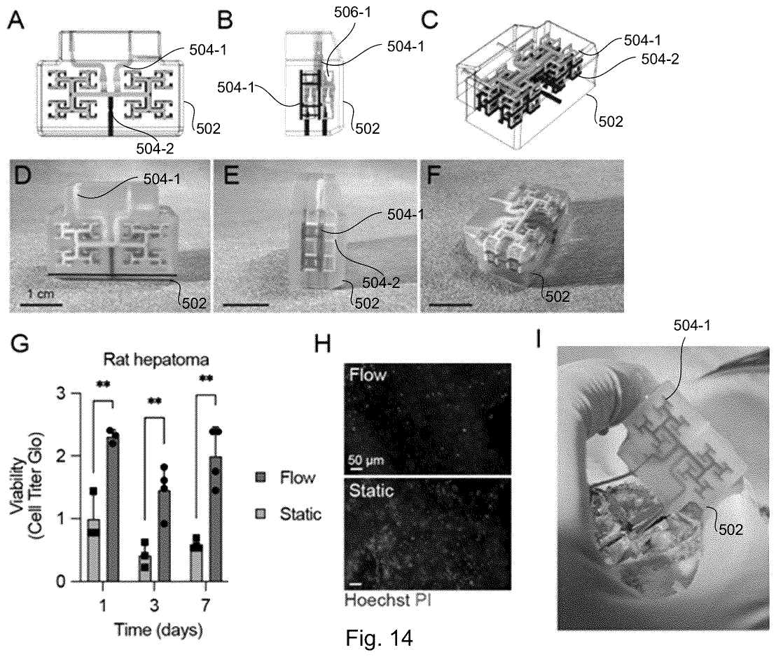

FIG. 1 depicts a block diagram of a distributed client-server system (e.g., distributed client-server system 100) according to some embodiments of the present disclosure. The system 100 facilitates fabricating a microchannel network device (e.g., microchannel network device 500-1 of FIG. 7, microchannel network device 500-2 of FIG. 14, etc.). Furthermore, the system 100 facilitates seeding and culturing a plurality of cells within the microchannel network device 500. In the way, the present disclosure provides systems and methods for fabricating and seeding cells within a microchannel network device 500, that otherwise was not previously feasible.

Other topologies of the system 100 are possible. For instance, in some embodiments, any of the illustrated devices and systems can in fact constitute several computer systems that are linked together in a network, or be a virtual machine or container in a cloud-computing environment. Moreover, rather than relying on a physical communication network 106, the illustrated devices and systems may wirelessly transmit information between each other. As such, the exemplary topology shown in FIG. 1 merely serves to describe the features of an embodiment of the present disclosure in a manner that will be readily understood to one of skill in the art.

Referring to FIG. 1, in some embodiments, a distributed client-server system 100 includes a fabrication system 200 for facilitating the fabrication of a microchannel network device 500, a system 200 for facilitating a culturing of cells within the microchannel network device 500, and one or more client devices 400 (e.g., a first client device 400-1), hereinafter “client device,”

In some embodiments, the communication network 106 optionally includes the Internet, one or more local area networks (LANs), one or more wide area networks (WANs), other types of networks, or a combination of such networks.

Examples of communication networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.1In), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

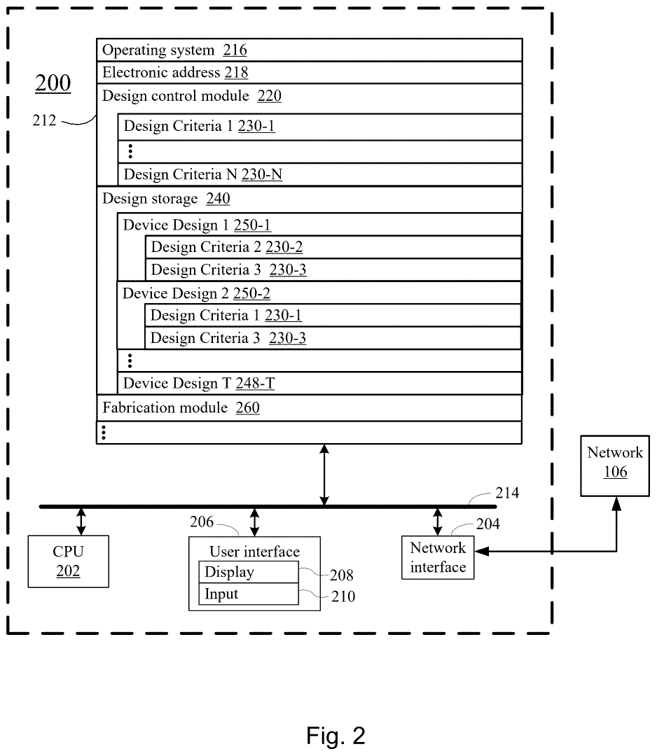

Now that a distributed client-server system 100 has generally been described, an exemplary fabrication system 200 for fabricating a microchannel network device 500 will be described with reference to FIG. 2.

In various embodiments, the fabrication system 200 includes one or more processing units (CPUs) 202, a network or other communications interface 204, and memory 212.

Memory 212 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 212 may optionally include one or more storage devices remotely located from the CPU(s) 202. Memory 212, or alternatively the non-volatile memory device(s) within memory 212, includes a non-transitory computer readable storage medium. Access to memory 212 by other components of the fabrication system 200, such as the CPU(s) 202, is, optionally, controlled by a controller. In some embodiments, memory 212 can include mass storage that is remotely located with respect to the CPU(s) 202. In other words, some data stored in memory 212 may in fact be hosted on devices that are external to the system 200, but that can be electronically accessed by the fabrication system 200 over an Internet, intranet, or other form of network 106 or electronic cable using communication interface 204.

In some embodiments, the memory 212 of the fabrication system 200 for generating a microchannel network device 500 stores:
an operating system 216 (e.g., ANDROID, iOS, DARWIN, RTXC, LINUX, UNIX, OS X, WINDOWS, or an embedded operating system such as VxWorks) that includes procedures for handling various basic system services;
an electronic address 218 associated with the fabrication system 200 that identifies the fabrication system 200;
a design control module 220 for forming a respective microchannel network device based one or more design criteria 230;
a design storage 240 for storing a plurality of designs 240, each of which is associated with a corresponding microchannel network device; and
a fabrication module 260 for facilitating a fabrication of a microchannel network device.

An electronic address 218 is associated with the system 200, which is utilized to at least uniquely identify the system 200 from other devices and components of the distributed system 100. For instance, in some embodiments, the electronic address 218 is utilized to receive a request from a client device 400 to generate and/or communicate a patient specific report 600.

A design control module 220 includes a plurality of design criteria 230, which for a basis for fabricating a respective microchannel network device. For instance, in some embodiments, a respective design criterion 230 in the plurality of design criteria 230 is associated with a parameter of the microchannel network device (e.g., a length of the microchannel network device, a porosity of a material forming the microchannel network device, etc.), a parameter of the fabrication system 200 (e.g., a resolution of the fabrication system 200, etc.), or both. In some embodiments, the plurality of design criteria 230 include one or more predetermined design criteria. In some embodiments, a predetermined design criteria includes a predetermined range of selections for a subject to choose from in fabrication a microchannel network device. In this way, the subject is preventing from selecting a design criteria that is outside a functional embodiment of a microchannel network device 500.

For instance, in some embodiments the plurality of design criteria are utilized and/or based from empirical parameters used to model a flow within a respective microchannel device. For instance, in some embodiments, the plurality of design criteria include a plurality of flow design criteria including a flow rate, a diffusion rate, or both (e.g., a diffusion rate during flow and/or a diffusion rate without flow).

A design storage 240 retains one or more microchannel network devices designs 250. Each respective design 250 includes a respective selection of one or more design criteria 230. In this way, a subject can select a design 250 from the design 240 without having to individually select respective design criteria 230 in order to fabricate a microchannel network device 500. For instance, in some embodiments, a subject determines a respective plurality of design criteria 230 to fabricate a corresponding microchannel vascular network. Accordingly, in response to determine a respective plurality of design criteria 230, the fabrication system 200 can retain a corresponding device design 250 that is associated with the corresponding microchannel vascular network. Thus, the subject can fabricate a plurality of the corresponding microchannel vascular network without having to determine the design criteria for each instance of the fabricating.

For instance, in some embodiments, the one or more device designs 250 includes a predetermined microchannel network device design 250 that is configured for a candidate subject. As a non-limiting example, in some embodiments, a first microchannel network device design is configured for a first mammalian candidate subject, and a second microchannel network device design is configured for a second mammalian candidate subject. In some embodiments, the first mammalian candidate subject and the second mammalian candidate subject are the same species of mammalian (e.g., both are configured for a human mammalian candidate subject, both are configured for a rat mammalian candidate subject, etc.). Furthermore, in some embodiments, the first mammalian candidate subject and the second mammalian candidate subject are the same species of mammalian but are different candidate subjects (e.g., the first candidate subject is a first human and the second candidate subject is a second human different than the first human). However, the present disclosure is not limited thereto. For instance, in some embodiments, the first the first mammalian candidate subject and the second mammalian candidate subject belong to different species of and/or a different genus of mammals.

As another non-limiting example, in some embodiments, the device designs 250 include one or more devices designs that is associated with an in vivo implementation, and one or more device designs that is associated with an in vitro implementation. For instance, in some embodiments, a first device design 250-1 is associated with a first microchannel network device including a first plurality of design criteria 230 associated with a first channel, a second channel, and a structure interposing between the first and second channels, a layer thickness of 50 μm, an exposure time of 2 seconds, a first layer time scale factor (FLTSF) of about 2× to about 4×, and a fabrication time of about 48 minutes. In some embodiments, a second device design 250-2 is associated with a second microchannel network device including a second plurality of design criteria 230 associated with a first channel, a second channel, and a structure interposing between the first and second channels, a layer thickness of 100 μm, an exposure time of 4 seconds, an FLTSF of about 2× to about 4×, and a fabrication time of about 24 minutes. In some embodiments, a third device design 250-3 is associated with a third microchannel network device including a third plurality of design criteria 230 associated with a first channel, a second channel, and a structure interposing between the first and second channels, a layer thickness of 100 μm, an exposure time of 4 seconds, an FLTSF of about 4× to about 8×, and a fabrication time of about 72 minutes to about 77 minutes. In some embodiments, the third device design 250-3 is associated with a rat mammalian candidate subject.

The fabrication system 200 includes a fabrication module 260 that facilitates controlling a mechanism for fabricating a microchannel network device. For instance, in some embodiments, the fabrication module communications one or more instructions to the mechanism, which, in response to the one or more instructions, traverses a region and conducts a fabrication of the microchannel network device. As a non-limiting example, consider a fabrication system 200 including a vat photopolymerization mechanism. Accordingly, the fabrication module 260 communications one or more instructions to the vat photopolymerization mechanism associated with an intensity of light associated with the vat photopolymerization mechanism, an exposure time of light associated with the vat photopolymerization mechanism, a supply of a pre-polymer solution of the vat photopolymerization mechanism, a supply of a photo initiator associated with the vat photopolymerization mechanism, and the like.

Each of the above identified modules and applications correspond to a set of executable instructions for performing one or more functions described above and the methods described in the present disclosure (e.g., the computer-implemented methods and other information processing methods described herein). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 212 optionally stores a subset of the modules and data structures identified above. Furthermore, in some embodiments, the memory 212 stores additional modules and data structures not described above.

It should be appreciated that the fabrication system 200 of FIG. 2 is only one example of a fabrication system 200, and that the fabrication system 200 optionally has more or fewer components than shown, optionally combines two or more components, or optionally has a different configured or arrangement of the components. The various components shown in FIG. 2 are implemented in hardware, software, firmware, or a combination thereof, including one or more signal processing and/or application specific integrated circuits.

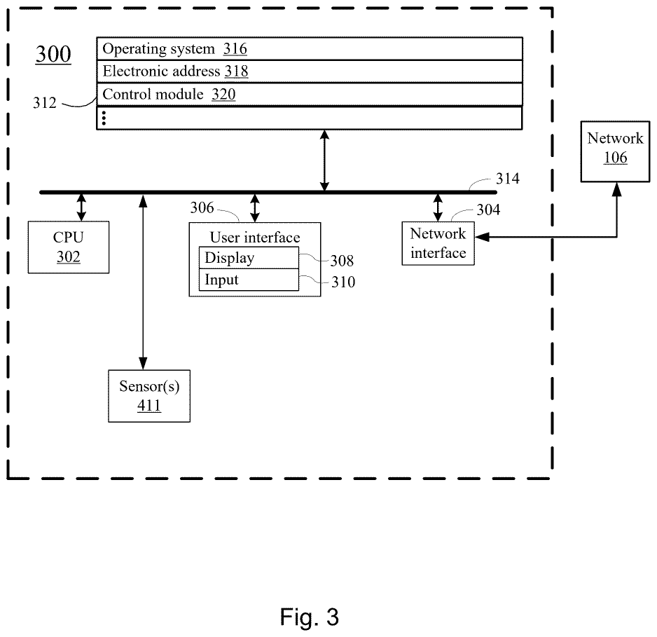

Referring to FIG. 3, in various embodiments, the present disclosure includes a bioreactor system 300. The bioreactor system 300 includes one or more processing units (CPUs) 302, a network or other communications interface 304, and memory 312.

Memory 312 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 312 may optionally include one or more storage devices remotely located from the CPU(s) 202.

Memory 312, or alternatively the non-volatile memory device(s) within memory 312, includes a non-transitory computer readable storage medium. Access to memory 312 by other components of the bioreactor system 300, such as the CPU(s) 302, is, optionally, controlled by a controller. In some embodiments, memory 312 can include mass storage that is remotely located with respect to the CPU(s) 302. In other words, some data stored in memory 312 may in fact be hosted on devices that are external to the bioreactor system 300, but that can be electronically accessed by the bioreactor system 300 over an Internet, intranet, or other form of network 106 or electronic cable using communication interface 304.

In some embodiments, the memory 312 of the bioreactor system 300 for culturing a plurality of cells within a microchannel network device 500 stores:
an operating system 316 (e.g., ANDROID, iOS, DARWIN, RTXC, LINUX, UNIX, OS X, WINDOWS, or an embedded operating system such as VxWorks) that includes procedures for handling various basic system services;
an electronic address 318 associated with the bioreactor system 300 that identifies the bioreactor system 300; and
a control module for controlling one or more system parameters of the bioreactor system 300.

An electronic address 318 is associated with the bioreactor system 300, which is utilized to at least uniquely identify the bioreactor system 300 from other devices and components of the distributed system 100. For instance, in some embodiments, the electronic address 318 is utilized to receive a design of a microchannel network device 500 from a client device 400.

A control module 320 facilitates communicating one or more instructions to a component of the bioreactor system 300. For instance, in some embodiments, the bioreactor system 300 includes a pump (e.g., pump 1720 of FIG. 17) and/or one or more reservoirs (e.g., reservoir(s) 1710 of FIG. 17), whose operation is controlled through the control module 320. For instance, in some embodiments, the control module 320 controls a flow rate of the bioreactor system 300.

Each of the above identified modules and applications correspond to a set of executable instructions for performing one or more functions described above and the methods described in the present disclosure (e.g., the computer-implemented methods and other information processing methods described herein). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 312 optionally stores a subset of the modules and data structures identified above. Furthermore, in some embodiments, the memory 312 stores additional modules and data structures not described above.

It should be appreciated that the bioreactor system 300 of FIG. 3 is only one example of a bioreactor system 300, and that the bioreactor system 300 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

Referring to FIG. 4, an exemplary client device 400 is provided (e.g., first client device 400-1). A client device 400 includes one or more processing units (CPUs) 402, one or more network or other communication interfaces 404, memory 411 (e.g., random access memory and/or non-volatile memory) optionally accessed by one or more controllers, and one or more controllers, and one or more communication busses 414 interconnecting the aforementioned components.

In some embodiments, a client device 400 includes a mobile device, such as a mobile phone, a tablet, a laptop computer, a wearable device such as a smart watch, and the like. Alternatively, in some embodiments, the client device 400 is a desktop computer or other similar devices. Furthermore, in some embodiments, the client devices 400 (e.g., a first user device 300-1, a second user device 400-2, a third user device 400-3, etc.) communicate with a centralized client device 400 (e.g., a server client device 400) that facilitates communicating a design of a microchannel network device 500 to the fabrication system 200.

In addition, the client device 400 includes a user interface 406. The user interface 406 typically includes a display device 408 for presenting media, such as a graphical user interface associated with a client application 420 and receiving instructions from the subject operating the client device 400. In some embodiments, the display device 408 is optionally integrated within the client device 400 (e.g., housed in the same chassis as the CPU 402 and memory 412), such as a smart (e.g., smart phone) device. In some embodiments, the client device 400 includes one or more input device(s) 410, which allow the subject to interact with the client device 400. In some embodiments, input devices 410 include a keyboard, a mouse, and/or other input mechanisms. Alternatively, or in addition, in some embodiments, the display device 408 includes a touch-sensitive surface, e.g., where display 408 is a touch-sensitive display or client device 400 includes a touch pad.

In some embodiments, the client device 400 includes an input/output (I/O) subsystem 430 for interfacing with one or more peripheral devices with the client device 400. For instance, in some embodiments, audio is presented through an external device (e.g., speakers, headphones, etc.) that receives audio information from the client device 400 and/or a remote device (e.g., fabrication system 200), and presents audio data based on this audio information. In some embodiments, the input/output (I/O) subsystem 430 also includes, or interfaces with, an audio output device, such as speakers or an audio output for connecting with speakers, earphones, or headphones. In some embodiments, the input/output (I/O) subsystem 430 also includes voice recognition capabilities (e.g., to supplement or replace an input device 410). In some embodiments, the input/output (I/O) subsystem 430 is configured to control one or more pumps.

In some embodiments, the client device 400 also includes one or more sensors 411 (e.g., an accelerometer, a magnetometer, a proximity sensor, a gyroscope, etc.), an image capture device (e.g., a camera device or an image capture module and related components), a location module (e.g., a Global Positioning System (GPS) receiver or other navigation or geolocation system module/device and related components), or a combination thereof, and the like. In some embodiments, the one or more sensors 411 is configured to control one or more pumps.

Memory 412 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 412 may optionally include one or more storage devices remotely located from the CPU(s) 402. Memory 412, or alternatively the non-volatile memory device(s) within memory 412, includes a non-transitory computer readable storage medium. Access to memory 412 by other components of the client device 400, such as the CPU(s) 402 and the I/O subsystem 330, is, optionally, controlled by a controller. In some embodiments, memory 412 can include mass storage that is remotely located with respect to the CPU 402. In other words, some data stored in memory 412 may in fact be hosted on devices that are external to the client device 400, but that can be electronically accessed by the client device 400 over an Internet, intranet, or other form of network 106 or electronic cable using communication interface 404.

In some embodiments, the memory 412 of the client device 400 stores:
an operating system 416 that includes procedures for handling various basic system services;
an electronic address 418 associated with the client device that identifies the client device; and
a client application 420 for generating content for display through a graphical user interface presented on the display 408 the client device 400.

An electronic address 418 is associated with the client device 400, which is utilized to at least uniquely identify the client device 400 from other devices and components of the distributed system 100. In some embodiments, the electronic address 418 associated with the client device 400 is used to determine a source of a communication received from and/or provided to the client device 400.

In some embodiments, a client application 420 is a group of instructions that, when executed by a processor (e.g., CPU(s) 402), generates content (e.g., a graphical user interface for selecting one or more design criteria of a microchannel network device 500) for presentation to the subject. In some embodiments, the client application 420 generates content in response to one or more inputs received from the subject through the user interface 406 of the client device 400. For instance, in some embodiments, the client application 420 includes a media presentation application for viewing the contents of a file or web application.

Each of the above identified modules and applications correspond to a set of executable instructions for performing one or more functions described above and the methods described in the present disclosure (e.g., the computer-implemented methods and other information processing methods described herein). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 312 optionally stores a subset of the modules and data structures identified above. Furthermore, in some embodiments, the memory 312 stores additional modules and data structures not described above.

It should be appreciated that the client device 400 of FIG. 4 is only one example of a client device 400, and that the client device 400 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

Various aspects of the present disclosure are directed to providing systems and methods for designing, fabricating, and/or utilizing an implantable a device. However, in some embodiments, a device 500 of the present disclosure includes an artificial organ device (e.g., an implant), a bioreactor device, a lab on a chip device (e.g., an organ on a chip device), and the like.

Accordingly, an exemplary device includes a body 502 that at least a first channel 504-1 and a second channel 504-2 (e.g., first intrinsic channel, second intrinsic channel, first channel network, second channel network, etc.), through which the medium flows. Further, in some embodiments each of the first channel and the second channel includes a first opening disposed at a first end portion of the device and a second opening. The respective second openings of the first channel and the second channel may be disposed on the same end portion of the device (e.g., a second end portion of the device) or on different end portions of the device (e.g., the second end portion of the device and a third end portion of the device, respectively). Thus, each opening of a respective channel acts as an inlet or an outlet for the flow of the medium. However, the present disclosure is not limited thereto. In some embodiments, a device of the present disclosure includes a master inlet and a master inlet that are in communication with the first channel and the second channel.

As previously described, in some embodiments an exemplary device is a component of a system, such as a bioreactor system. Thus, in some embodiments each opening of a respective channel includes a portion for facilitating coupling to another component of the system, such as tubing in communication with a reservoir of the medium or a pump 2210. In some embodiments, the coupling portion of each respective channel includes a coupling diameter that is different from a channel diameter of the respective channel. In some embodiments, the first opening and the second opening of the first channel and the second channel each have a channel diameter in a range of from about 500 micrometers (μm) to about 10,000 μm, about 500 μm to about 5,000 μm, about 500 μm to about 2,500 μm, about 500 μm to about 1,500 μm, about 500 μm to about 1,000 μm, about 650 μm to about 2,500 μm, about 650 μm to about 1,500 μm, about 650 μm to about 1,000 μm, or about 650 μm to about 750 μm. Furthermore, in some embodiments the first opening and the second opening of the first channel have a different diameter than the first opening and the second opening of the second channel.

In some embodiments, the body 502 of the device includes a hydrogel material. For instance, in some embodiments, GelMA is synthesized by a direct reaction of gelatin with methacrylic anhydride (MA) in the presence of a buffer. This reaction introduces methacryloyl substitution groups on the reactive amine and hydroxyl groups of the amino acid residues (FIG. 2). Different degrees of methacryloyl substitution are achievable in GelMA by tuning the amount of MA added to the reaction mixture, which produces GelMA with different physical properties. Photo-crosslinking of the synthesized GelMA can be conducted under exposure to ultraviolet (UV) light using a water-soluble initiator. The degree of substitution, a GelMA concentration, a photo-initiator (PI) concentration, and an exposure time to UV light are some of the parameters that allow tuning of the physical properties of the resulting GelMA hydrogel body 502. However, the present disclosure is not limited thereto.

In some embodiments, a first channel network 504-1 is formed in the body 502. In some embodiments, the first channel network 504-1 is configured to receive cells, such as a first plurality of viable cells. For instance, in some embodiments, the first channel network 504-1 includes a first set of one or more channels, such as a first channel, and the second channel network 504-2 includes a second set of one or more channels, such as a second channel different from the first channel. In some embodiments, the second channel network 504-2 is formed recursively from the first channel network 504-1.

In some embodiments, each channel network includes an inlet and one or more channels in fluidic communication with the inlet. In some embodiments, the one or more channels is formed in a series of branching (e.g., bifurcating) channels, with each branch producing two or more channels of a smaller size in fluidic communication with a parent channel that forms a respective branch. In some embodiments, the devices includes more than one channel network (e.g., a first channel network and a second channel network) that are in fluidic communication with one another. In some embodiments, branching of the channels occurs in a linear tree. In some embodiments, branching occurs in a radial tree.

In some embodiments, the first intrinsic channel network includes one or more first fractal architectures formed by the branching of the channels. For instance, in some embodiments, the branching of the channels forms one or more generations of first channel junctions, in which a channel bifurcates into two subsequent channels. In some embodiments, the second intrinsic channel network includes one or more second fractal architectures formed by the branching of the channels. For instance, in some embodiments, each respective first fractal architecture in the one or more first fractal architectures is adjacent to a corresponding second fractal architecture in the one or more second fractal architectures, which collectively form a corresponding channel layer in one or more channel layers of the device. However, the present disclosure is not limited thereto.

In some embodiments, the one or more channels is formed in a variety of shapes and corresponding cross-sections including, but not limited to, a circular cross-section, a rectangular cross-section, or a corresponding cross-section of a platonic solid. In some embodiments, an aspect ratio of a cross-section of each channel in a channel network is uniform (e.g., a uniform aspect ratio of 1:1). In some embodiments, an aspect ratio of a cross-section of each channel in a channel network is uniform except at a portion of a connector.

In some embodiments, the channel and/or the inlet of a respective channel network is formed intrinsic with the body 502 of the device. For instance, in some embodiments, the intrinsic channel network is integrally formed with the body of the device, such that the body of the device is a three-dimensional monolithic feature. However, the present disclosure is not limited thereto. In some embodiments, the intrinsic channel network is integrally formed with the inlet of the body and the outlet of the body, such that the intrinsic channel network.

Moreover, in some embodiments, the barrier is formed concurrently with the device through additive manufacturing (e.g., 3D printing), such that the barrier is configurable to control flow paths within and/or surrounding the device. In some embodiments, the barrier has a thickness in a range of from 5 μm to 10,000 μm, from 5 μm to 5,000 μm, from 10 μm to 5,000 μm, or from 10 μm to 4,000 μm. In some embodiments, the barrier includes a plurality of pores. In some embodiments, a pore size of the barrier is smaller than a diameter of a cell in a respective plurality of viable cells configured to be received by a corresponding channel of the device. Accordingly, the cells will not be able to pass through the barrier (e.g., a low permeability for animal cells), while low molecular weight nutrients and fluids pass through (i.e. a high permeability for nutrients), providing adequate cell-to-cell signaling. In some embodiments, cell sizes vary, and in general are in a range of microns. For example, in some embodiments, a red blood cell has a diameter of approximately 8 μm. Preferably, the average pore size of the barrier is on a submicron-scale to ensure effective screening of the cells. In some embodiments, a permeability of the barrier is determined by a number of parameters including a property of the barrier (e.g., a pore size and/or a porosity), an interaction and/or an affinity between the barrier and a material, a size of a cell species, a concentration gradient of a material, an elasticity of a material, and/or a combination thereof. In some embodiments, a distance from a center of a first pore to a center of an adjacent pore of the barrier is in a range of from 5 μm to 150 μm, from 5 μm to 100 μm, or from 5 μm to 50 μm. In some embodiments, a diameter of each pore is in a range of from 5 μm to 150 μm, from 5 μm to 100 μm, or from 5 μm to 50 μm. Furthermore, in some embodiments, the depth of each pore is in a range of from 5 μm to 5,000 μm, from 10 μm to 5,000 μm, from 10 μm to 4,000 μm, or from 10 μm to 1,000 μm. In some embodiments, a pore has a rectangular shape (e.g., a rectangular opening and/or cross-section), a square shape, a cylindrical shape, a conical shape, a cup shape, an hourglass, or the like. In some embodiments, the barrier includes a material with a non-zero solubility to a predetermined solution or chemical. In some embodiments, the barrier includes a polymer with a high permeability for a predetermined solution or chemical. In some embodiments, the barrier includes polydimethylsiloxane, which has a high permeability to fluids such as oxygen and carbon dioxide. However, the present disclosure is not limited thereto.

In some embodiments, a second intrinsic channel network 504-2 is formed in the body 502. In some embodiments, the second channel network 504-2 is configured to allow for active flow of a fluid to support viability and/or function of the cells in the first intrinsic channel network 504-1. However, the present disclosure is not limited thereto. In some at least a segment of the first channel and a segment of the second channel are parallel to each other and spaced apart from each other at a first distance. For instance, in some embodiments, an edge portion of the first channel and an edge portion of the second channel do not interface, such that the first distance separates the edge portion of the first channel and the edge portion of the second channel. However, the present disclosure is not limited thereto. For instance, in some embodiments a thickness of the walls of the channels is modified by altering a cross-section of the channels. In some embodiments, a wall thickness of each surface of a channel is the same thickness. In some embodiments, a wall thickness of each surface of a channel is in a range of from 5 μm to 10 millimeters (mm), from 5 μm to 1,000 μm, from 5 μm to 500 μm, or from 10 μm to 500 μm. Furthermore, in some embodiments a distance between a surface of a channel and a nearest adjacent channel surface (e.g., a void, a dead volume) is in a range of from 5 μm to 5 cm, from 5 μm to 4 cm, from 10 μm to 4 cm, from 10 μm to 1 cm, from 10 μm to 1 cm, or from 10 μm to 1,000 μm. However, the present disclosure is not limited thereto.

In some embodiments, the body forms a first barrier 506-1 that allows for fluidic communication between the first channel and the second channel to nourish the cells. For instance, in some embodiments, the first barrier is formed between the segment of the first channel and the segment of the second channel. In some embodiments, the barrier is formed between adjacent portions of parallel channels of the device. In some embodiments, the barrier includes a porosity or a pore density as a fraction of an area and/or a thickness of the barrier. In some embodiments, the thickness of the barrier is the same as the first length. However, the present disclosure is not limited thereto.

In some embodiments, the barrier is configured to selectively allow a flow of material from the second channel network to the first channel network and/or from the first channel network to the second channel network. In some embodiments, the barrier is a membrane, such as a hydrogel membrane having a porosity of a first dimension, such as a first nanoscale dimension. However, the present disclosure is not limited thereto.

In some embodiments, the first distance between the segment of the first channel and the segment of the second channel is from about 10 μm to about 1000 μm. In some embodiments, the first distance is about 300 μm.

In some embodiments, the first distance between the segment of the first channel and the segment of the second channel is at least 10 microns, at least 60 microns, at least 115 microns, at least 165 microns, at least 220 microns, at least 270 microns, at least 300 microns, at least 375 microns, at least 425 microns, at least 480 microns, at least 530 microns, at least 585 microns, at least 635 microns, at least 685 microns, at least 740 microns, at least 790 microns, at least 845 microns, at least 895 microns, at least 950 microns, or at least 1000 microns.

In some embodiments, the first distance between the segment of the first channel and the segment of the second channel is at most 10 microns, at most 60 microns, at most 115 microns, at most 165 microns, at most 220 microns, at most 270 microns, at most 300 microns, at most 375 microns, at most 425 microns, at most 480 microns, at most 530 microns, at most 585 microns, at most 635 microns, at most 685 microns, at most 740 microns, at most 790 microns, at most 845 microns, at most 895 microns, at most 950 microns, or at most 1000 microns.

In some embodiments, the first distance between the segment of the first channel and the segment of the second channel is in a range that contains between 10 microns and 60 microns, between 10 microns and 270 microns, between 10 microns and 480 microns, between 10 microns and 740 microns, between 10 microns and 950 microns, between 10 microns and 1000 microns, between 60 microns and 270 microns, between 60 microns and 530 microns, between 60 microns and 740 microns, between 60 microns and 950 microns, between 60 microns and 1000 microns, between 115 microns and 300 microns, between 115 microns and 530 microns, between 115 microns and 790 microns, between 115 microns and 1000 microns, between 165 microns and 220 microns, between 165 microns and 425 microns, between 165 microns and 685 microns, between 165 microns and 895 microns, between 165 microns and 1000 microns, between 220 microns and 375 microns, between 220 microns and 585 microns, between 220 microns and 790 microns, between 220 microns and 1000 microns, between 270 microns and 375 microns, between 270 microns and 585 microns, between 270 microns and 790 microns, between 270 microns and 1000 microns, between 300 microns and 425 microns, between 300 microns and 635 microns, between 300 microns and 845 microns, between 300 microns and 1000 microns, between 375 microns and 480 microns, between 375 microns and 685 microns, between 375 microns and 950 microns, between 375 microns and 1000 microns, between 425 microns and 635 microns, between 425 microns and 845 microns, between 425 microns and 1000 microns, between 480 microns and 635 microns, between 480 microns and 845 microns, between 480 microns and 1000 microns, between 530 microns and 635 microns, between 530 microns and 845 microns, between 530 microns and 1000 microns, between 585 microns and 685 microns, between 585 microns and 950 microns, between 585 microns and 1000 microns, between 635 microns and 845 microns, between 635 microns and 1000 microns, between 685 microns and 790 microns, between 685 microns and 1000 microns, between 740 microns and 845 microns, between 740 microns and 1000 microns, between 790 microns and 895 microns, between 790 microns and 1000 microns, between 845 microns and 1000 microns, between 895 microns and 950 microns, between 895 microns and 1000 microns, or between 950 microns and 1000 microns, inclusive.

In some embodiments, the first channel has a nominal volume from about 1 μL to about 1000 μL. In some embodiments, the nominal volume is about 20 μL.

In some embodiments, the nominal volume is at least 20 μL, at least 55 μL, at least 105 μL, at least 160 μL, at least 210 μL, at least 265 μL, at least 315 μL, at least 370 μL, at least 420 μL, at least 475 μL, at least 525 μL, at least 580 μL, at least 630 μL, at least 685 μL, at least 735 μL, at least 790 μL, at least 840 μL, at least 895 μL, at least 945 μL, or at least 1000 μL.

In some embodiments, the nominal volume is at most 20 μL, at most 55 μL, at most 105 μL, at most 160 μL, at most 210 μL, at most 265 μL, at most 315 μL, at most 370 μL, at most 420 μL, at most 475 p L, at most 525 μL, at most 580 μL, at most 630 μL, at most 685 μL, at most 735 μL, at most 790 μL, at most 840 μL, at most 895 μL, at most 945 μL, or at most 1000 μL.

In some embodiments, the nominal volume is in a range that contains between 20 μL and 55 μL, between 20 μL and 265 μL, between 20 μL and 475 μL, between 20 μL and 735 μL, between 20 μL and 945 μL, between 20 μL and 1000 μL, between 55 μL and 265 μL, between 55 μL and 525 μL, between 55 μL and 735 μL, between 55 μL and 945 μL, between 55 μL and 1000 μL, between 105 μL and 315 μL, between 105 μL and 525 μL, between 105 μL and 790 μL, between 105 μL and 1000 μL, between 160 μL and 210 μL, between 160 μL and 420 μL, between 160 μL and 685 μL, between 160 μL and 895 μL, between 160 μL and 1000 μL, between 210 μL and 370 μL, between 210 μL and 580 μL, between 210 μL and 790 μL, between 210 μL and 1000 μL, between 265 μL and 370 μL, between 265 μL and 580 μL, between 265 μL and 790 μL, between 265 μL and 1000 μL, between 315 μL and 420 μL, between 315 μL and 630 μL, between 315 μL and 840 μL, between 315 μL and 1000 μL, between 370 μL and 475 μL, between 370 μL and 685 μL, between 370 μL and 945 μL, between 370 μL and 1000 μL, between 420 μL and 630 μL, between 420 μL and 840 μL, between 420 μL and 1000 μL, between 475 μL and 630 μL, between 475 μL and 840 μL, between 475 μL and 1000 μL, between 525 μL and 630 μL, between 525 μL and 840 μL, between 525 μL and 1000 μL, between 580 μL and 685 μL, between 580 μL and 945 μL, between 580 μL and 1000 μL, between 630 μL and 840 μL, between 630 μL and 1000 μL, between 685 μL and 790 μL, between 685 μL and 1000 μL, between 735 μL and 840 μL, between 735 μL and 1000 μL, between 790 μL and 895 μL, between 790 μL and 1000 μL, between 840 μL and 1000 μL, between 895 μL and 945 μL, between 895 μL and 1000 μL, or between 945 μL and 1000 μL, inclusive.

In some embodiments, the segment of the first channel or the segment of the second channel has a length of from about 1 mm to about 100 mm. In some embodiments, the length is about 12 mm.

In some embodiments, the length is at least 1 mm, at least 5 mm, at least 10 mm, at least 12 mm, at least 15 mm, at least 25 mm, at least 30 mm, at least 35 mm, at least 40 mm, at least 45 mm, at least 50 mm, at least 55 mm, at least 60 mm, at least 65 mm, at least 70 mm, at least 80 mm, at least 85 mm, at least 90 mm, at least 95 mm, or at least 100 mm.

In some embodiments, the length is at most 1 mm, at most 5 mm, at most 10 mm, at most 12 mm, at most 15 mm, at most 25 mm, at most 30 mm, at most 35 mm, at most 40 mm, at most 45 mm, at most 50 mm, at most 55 mm, at most 60 mm, at most 65 mm, at most 70 mm, at most 80 mm, at most 85 mm, at most 90 mm, at most 95 mm, or at most 100 mm.

In some embodiments, the length is in a range that contains between 1 mm and 5 mm, between 1 mm and 25 mm, between 1 mm and 45 mm, between 1 mm and 70 mm, between 1 mm and 95 mm, between 1 mm and 100 mm, between 5 mm and 25 mm, between 5 mm and 50 mm, between 5 mm and 70 mm, between 5 mm and 95 mm, between 5 mm and 100 mm, between 10 mm and 30 mm, between 10 mm and 50 mm, between 10 mm and 80 mm, between 10 mm and 100 mm, between 12 mm and 15 mm, between 12 mm and 40 mm, between 12 mm and 65 mm, between 12 mm and 90 mm, between 12 mm and 100 mm, between 15 mm and 35 mm, between 15 mm and 55 mm, between 15 mm and 80 mm, between 15 mm and 100 mm, between 25 mm and 35 mm, between 25 mm and 55 mm, between 25 mm and 80 mm, between 25 mm and 100 mm, between 30 mm and 40 mm, between 30 mm and 60 mm, between 30 mm and 85 mm, between 30 mm and 100 mm, between 35 mm and 45 mm, between 35 mm and 65 mm, between 35 mm and 95 mm, between 35 mm and 100 mm, between 40 mm and 60 mm, between 40 mm and 85 mm, between 40 mm and 100 mm, between 45 mm and 60 mm, between 45 mm and 85 mm, between 45 mm and 100 mm, between 50 mm and 60 mm, between 50 mm and 85 mm, between 50 mm and 100 mm, between 55 mm and 65 mm, between 55 mm and 95 mm, between 55 mm and 100 mm, between 60 mm and 85 mm, between 60 mm and 100 mm, between 65 mm and 80 mm, between 65 mm and 100 mm, between 70 mm and 85 mm, between 70 mm and 100 mm, between 80 mm and 90 mm, between 80 mm and 100 mm, between 85 mm and 100 mm, between 90 mm and 95 mm, between 90 mm and 100 mm, or between 95 mm and 100 mm, inclusive.

In some embodiments, the body has a width of from about 5 mm to about 100 mm. In some embodiments, the width is about 25 mm.

In some embodiments, the width is at least 5 mm, at least 10 mm, at least 12 mm, at least 15 mm, at least 25 mm, at least 30 mm, at least 35 mm, at least 40 mm, at least 45 mm, at least 50 mm, at least 55 mm, at least 60 mm, at least 65 mm, at least 70 mm, at least 80 mm, at least 85 mm, at least 90 mm, at least 95 mm, or at least 100 mm.

In some embodiments, the width is at most 5 mm, at most 10 mm, at most 12 mm, at most 15 mm, at most 25 mm, at most 30 mm, at most 35 mm, at most 40 mm, at most 45 mm, at most 50 mm, at most 55 mm, at most 60 mm, at most 65 mm, at most 70 mm, at most 80 mm, at most 85 mm, at most 90 mm, at most 95 mm, or at most 100 mm.

In some embodiments, the width is in a range that contains between 5 mm and 25 mm, between 5 mm and 50 mm, between 5 mm and 70 mm, between 5 mm and 95 mm, between 5 mm and 100 mm, between 10 mm and 30 mm, between 10 mm and 50 mm, between 10 mm and 80 mm, between 10 mm and 100 mm, between 12 mm and 15 mm, between 12 mm and 40 mm, between 12 mm and 65 mm, between 12 mm and 90 mm, between 12 mm and 100 mm, between 15 mm and 35 mm, between 15 mm and 55 mm, between 15 mm and 80 mm, between 15 mm and 100 mm, between 25 mm and 35 mm, between 25 mm and 55 mm, between 25 mm and 80 mm, between 25 mm and 100 mm, between 30 mm and 40 mm, between 30 mm and 60 mm, between 30 mm and 85 mm, between 30 mm and 100 mm, between 35 mm and 45 mm, between 35 mm and 65 mm, between 35 mm and 95 mm, between 35 mm and 100 mm, between 40 mm and 60 mm, between 40 mm and 85 mm, between 40 mm and 100 mm, between 45 mm and 60 mm, between 45 mm and 85 mm, between 45 mm and 100 mm, between 50 mm and 60 mm, between 50 mm and 85 mm, between 50 mm and 100 mm, between 55 mm and 65 mm, between 55 mm and 95 mm, between 55 mm and 100 mm, between 60 mm and 85 mm, between 60 mm and 100 mm, between 65 mm and 80 mm, between 65 mm and 100 mm, between 70 mm and 85 mm, between 70 mm and 100 mm, between 80 mm and 90 mm, between 80 mm and 100 mm, between 85 mm and 100 mm, between 90 mm and 95 mm, between 90 mm and 100 mm, or between 95 mm and 100 mm, inclusive.

In some embodiments, the device further includes a plurality of ports 510 formed at a boundary of the body 502 and in fluidic communication with the first or second intrinsic channel network 504. In some embodiments, each respective port 510 in the plurality of ports 510 is configured to couple with a corresponding tube 514 in a plurality of tubes 514, which, as a non-limiting example, allows the implantable device to anastomose with a pump or host vasculature 2210 upon implantation. Accordingly, in some embodiments, each respective port 510 allows for ingress or egress of a fluidic medium via the channel of the device. However, the present disclosure is not limited thereto.

In some embodiments, the pump 2210 is peristaltic or osmotic. In some embodiments, the pump 2210 is configured to engage with one or more channels regulate flow rate of medium through the one or more channels. For instance, in some embodiments, the peristaltic constrain the flow in the one or more channels by interfacing with an exterior surface of a first channel or tube 514 of the device in fluidic communication with the one or more channels. However, the present disclosure is not limited thereto.

In some embodiments, the plurality of ports 510 is configured to maintain a pressure at a surface or interior of the channel at a threshold strain, such as a first threshold maximum principal strain of the body, such as in order to prevent the body from cracking.

In some embodiments, the plurality of ports 510 includes a first port in fluidic communication with a first end of the second intrinsic channel network and configured to couple with a first tube in the plurality of tubes 514 to receive the fluid for the second intrinsic channel network. In some embodiments, the plurality of ports 510 includes a second port in fluidic communication with a second end of the second intrinsic channel network and configured to couple with a second tube in the plurality of tubes to remove the fluid from the second intrinsic channel network.

In some embodiments, the first and second ports 510 are configured such that connection between the first port and the first tube and connection between the second port and the second tube are stable in excess of a systolic pressure of about 300 mmHg in absence of an adhesive. In some embodiments, the first and second ports 510 are configured such that connection between the first port and the first tube and connection between the second port and the second tube are each stable when a subject to a threshold pressure of about 300 mmHg, in which the first port and the first tube are coupled using a friction fit and/or a thermal fit (e.g., the first port and the first tube lack adhesive to bond the first port and the first tube together). However, the present disclosure is not limited thereto.

In some embodiments, the threshold pressure is at least 200 mmHg, at least 210 mmHg, at least 220 mmHg, at least 230 mmHg, at least 240 mmHg, at least 255 mmHg, at least 265 mmHg, at least 275 mmHg, at least 285 mmHg, at least 300 mmHg, at least 305 mmHg, at least 315 mmHg, at least 325 mmHg, at least 335 mmHg, at least 345 mmHg, at least 360 mmHg, at least 370 mmHg, at least 380 mmHg, at least 390 mmHg, or at least 400 mmHg.

In some embodiments, the threshold pressure is at most 200 mmHg, at most 210 mmHg, at most 220 mmHg, at most 230 mmHg, at most 240 mmHg, at most 255 mmHg, at most 265 mmHg, at most 275 mmHg, at most 285 mmHg, at most 300 mmHg, at most 305 mmHg, at most 315 mmHg, at most 325 mmHg, at most 335 mmHg, at most 345 mmHg, at most 360 mmHg, at most 370 mmHg, at most 380 mmHg, at most 390 mmHg, or at most 400 mmHg.

In some embodiments, the threshold pressure is between 200 mmHg and 210 mmHg, between 200 mmHg and 255 mmHg, between 200 mmHg and 300 mmHg, between 200 mmHg and 345 mmHg, between 200 mmHg and 390 mmHg, between 200 mmHg and 400 mmHg, between 210 mmHg and 255 mmHg, between 210 mmHg and 305 mmHg, between 210 mmHg and 345 mmHg, between 210 mmHg and 390 mmHg, between 210 mmHg and 400 mmHg, between 220 mmHg and 265 mmHg, between 220 mmHg and 305 mmHg, between 220 mmHg and 360 mmHg, between 220 mmHg and 400 mmHg, between 230 mmHg and 240 mmHg, between 230 mmHg and 285 mmHg, between 230 mmHg and 335 mmHg, between 230 mmHg and 380 mmHg, between 230 mmHg and 400 mmHg, between 240 mmHg and 275 mmHg, between 240 mmHg and 315 mmHg, between 240 mmHg and 360 mmHg, between 240 mmHg and 400 mmHg, between 255 mmHg and 275 mmHg, between 255 mmHg and 315 mmHg, between 255 mmHg and 360 mmHg, between 255 mmHg and 400 mmHg, between 265 mmHg and 285 mmHg, between 265 mmHg and 325 mmHg, between 265 mmHg and 370 mmHg, between 265 mmHg and 400 mmHg, between 275 mmHg and 300 mmHg, between 275 mmHg and 335 mmHg, between 275 mmHg and 390 mmHg, between 275 mmHg and 400 mmHg, between 285 mmHg and 325 mmHg, between 285 mmHg and 370 mmHg, between 285 mmHg and 400 mmHg, between 300 mmHg and 325 mmHg, between 300 mmHg and 370 mmHg, between 300 mmHg and 400 mmHg, between 305 mmHg and 325 mmHg, between 305 mmHg and 370 mmHg, between 305 mmHg and 400 mmHg, between 315 mmHg and 335 mmHg, between 315 mmHg and 390 mmHg, between 315 mmHg and 400 mmHg, between 325 mmHg and 370 mmHg, between 325 mmHg and 400 mmHg, between 335 mmHg and 360 mmHg, between 335 mmHg and 400 mmHg, between 345 mmHg and 370 mmHg, between 345 mmHg and 400 mmHg, between 360 mmHg and 380 mmHg, between 360 mmHg and 400 mmHg, between 370 mmHg and 400 mmHg, between 380 mmHg and 390 mmHg, between 380 mmHg and 400 mmHg, or between 390 mmHg and 400 mmHg, inclusive.

In some embodiments, the plurality of ports 510 includes a third port in fluidic communication with the first intrinsic channel network. In some embodiments, the third port is configured to drain, or egress, a product produced by the cells. In some embodiments, the third port is configured to drain, or egress, the medium after flowing through the first intrinsic channel network and/or the second intrinsic channel network.

In some embodiments, the plurality of ports 510 further includes a fourth port in fluidic communication with the second intrinsic channel network to assist in flowing the fluid in the second intrinsic channel network. In some embodiments, the fourth port is configured to receive, or ingress, the medium for flowing through the first intrinsic channel network and/or the second intrinsic channel network.

In some embodiments, the one or more channel layers receive the medium from a bifurcating distributor channel network (e.g., a first manifold) and provide or egress the medium via a bifurcating collector channel network (e.g., a second manifold different from the first manifold). In some embodiments, the one or more first fractal architectures and the one or more second fractal architectures are fluidly connected into a single fluidic system via the first manifold and the second manifold. However, the present disclosure is not limited thereto.

In some embodiments, the each of the one or more first fractal architectures comprises a plurality of generations of first channel T-junctions, and each of one or more second fractal architectures comprises a plurality of generations of second channel T-junctions.

In some embodiments, the first channel T-junctions at each generation are identical; channel segments connecting first channel T-junctions between two generations are identical; second channel T-junctions at each generation are identical; and channel segments connecting second channel T-junctions between two generations are identical.

In some embodiments, the first intrinsic channel network comprises a plurality of first fractal architectures and the second intrinsic channel network comprises a plurality of second fractal architectures, thereby forming a plurality of channel layers stacked together.

In some embodiments, the first fractal architectures in the plurality of first fractal architectures are identical to each other, and second fractal architectures in the plurality of second fractal architectures are identical to each other.

### EXAMPLES

### 3D Printed Device (3DPD) Development.

A fundamental functional structure of the 3D printed device (3DPD) design is a pair of parallel channels: one channel allows for active flow of blood or medium and the other contains cells. These two compartments simulate a blood vessel and a surrounding parenchymal space. To achieve a physical barrier with chemical permeability, the present disclosure selected polyethylene(glycol) diacrylate (PEGDA) as the scaffold material due to its printability, polymer availability and uniformity, low immunogenicity, relative mechanical strength, and flexibility for chemical modification. A PEGDA molecular weight of 700 Da was selected to produce a relatively high crosslink density to ensure mechanical strength while maintaining high (80%) porosity (FIG. 15A).

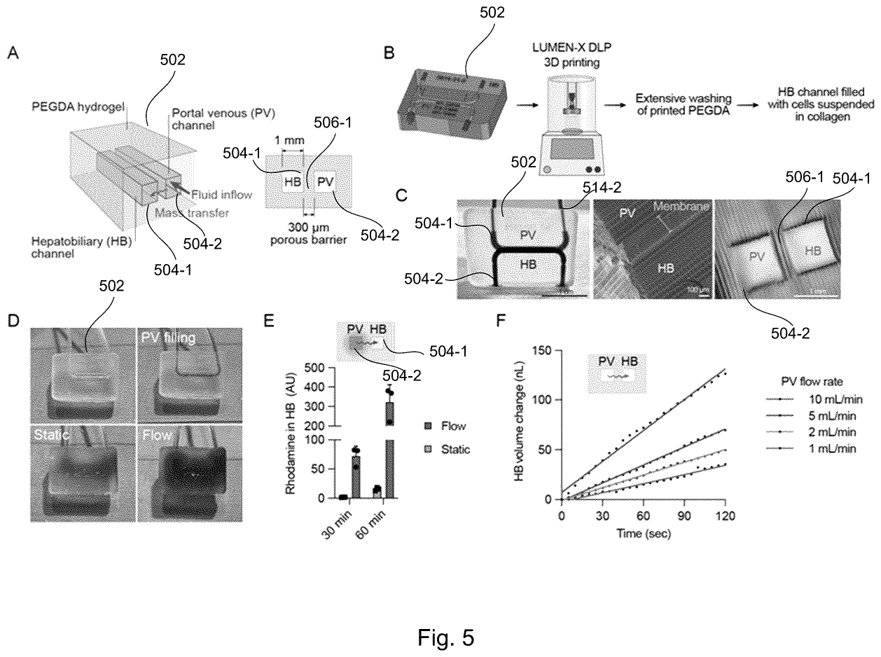

The basic structural motif of the 3DPD is thus two channels separated by a permeable PEGDA barrier, which allows fluid and solute movement between channels, in which the channels having a diameter that is micron to millimeter size. The present disclosure selected liver as an application; as such, one channel recapitulates portal venous (PV) blood flow, the other hepatobiliary (HB) (FIG. 5A).

3DPDs were 3D printed using a LUMEN-X digital light projection (DLP) printer, which is a modality ideally suited for fidelity printing of channels having a diameter that is micron to millimeter size. A PEGDA scaffold has minimal ability to interact directly with cells due to lack of bioactive chemical structures. Therefore, cells were suspended in a collagen matrix prior to loading into the HB channel and allowed to gel, resulting in a dense suspension of cells in a 3D collagen matrix within the PEGDA hydrogel (FIG. 5B).

Channels were printed with 1 mm square cross-sections separated by a 300 μm PEGDA porous barrier; the nominal volume of the HB channel was about 20 μL. The intersecting region of the PV and HB channels was about 12 mm long with a total device width of about 25 mm (FIG. 5C and FIG. 16A-16C).

To connect the 3DPD to external systems (in vitro bioreactors and in vivo vessels), ports for connecting silicone tubing were added to the channel termini. The present disclosure performed mechanical testing of 3D printed PEGDA dogbone geometries to determine strength in compression and tension. As expected, tension applied normal to 3D printed layers led to failure earlier than tension applied parallel to layers (FIG. 17A-17D). The measured material properties were used in a finite element model of the hydrogel port-tube interface which simulated the strain and hoop stress in the hydrogel resulting from the tube overfit—i.e. the tube outer diameters were slightly larger than the port diameter to achieve a snug fit (FIG. 17A-17H). By keeping the overfit—induced strain below the strain limit and adding additional wall thickness, the present disclosure generated optimized port dimensions to allow tubing to couple into the ports while keeping the maximum principal strain in the hydrogel as low as possible to prevent cracking. Due to the low molecular weight of PEGDA, volumetric swelling was limited (FIG. 15B), and post-print port dimensions were similar to post-swelling dimensions, advantageous for optimizing parameters.

With an optimized port/tube design, the connections were stable well in excess of human systolic pressure (~300 mmHg) without application of adhesives (FIG. 17I-17K). Additionally, the HB-PV channel barrier maintained integrity during stringent tube/hydrogel burst pressure testing (FIG. 17L). This design was sufficient to provide stable connections for in vitro systems and subsequent in vivo implantation.

Flow enhances chemical transport within the PEGDA hydrogel; as a demonstration, diffusion of red dye into the hydrogel was enhanced when perfused through the PV channel relative to static conditions (FIG. 5D). When the PV channel was filled with a solution containing the dye rhodamine, it was readily detected in the HB channel (FIG. 5E). Because perfusion of the PV channel maintains a constant high concentration of rhodamine, the difference in total diffusion between static and flow conditions increased with increased time. The present disclosure also found that a measurable pressure-driven flow could be produced between the PV and HB (FIG. 5F). This 3DPD design and material allowed for the mechanical robustness needed for surgical handling while allowing for perfusion between channels having a diameter that is micron to millimeter size to support cultured cells.

### Perfusion Enhances Viability and Function of Rat Hepatocytes.

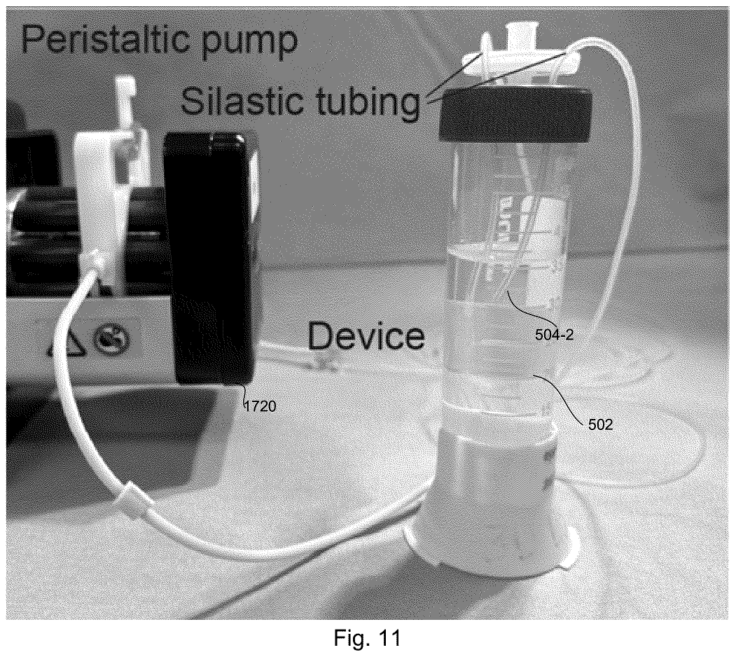

A custom bioreactor system composed of gas-permeable silastic tubing, modified polypropylene tubes, and a peristaltic pump was developed to evaluate in vitro the ability of 3DPDs to maintain cell viability and essential functions (FIG. 11). 3DPDs were loaded with a high density (20 million cells/mL) of rat hepatoma cells suspended in a collagen gel. Titrated cell densities in 3D collagen gels were cultured in static conditions in glass-bottom dishes to determine the concentration limit beyond which viability is affected. A density higher than this threshold was purposely chosen to provide a challenging environment by which to evaluate potential benefit of perfusion (FIG. 18A-18D). Due to the printed size, 3DPDs required multiple days of washing to remove unpolymerized pre-polymer components; once completed, the PEGDA was found to be biocompatible (FIG. 18E-18G).

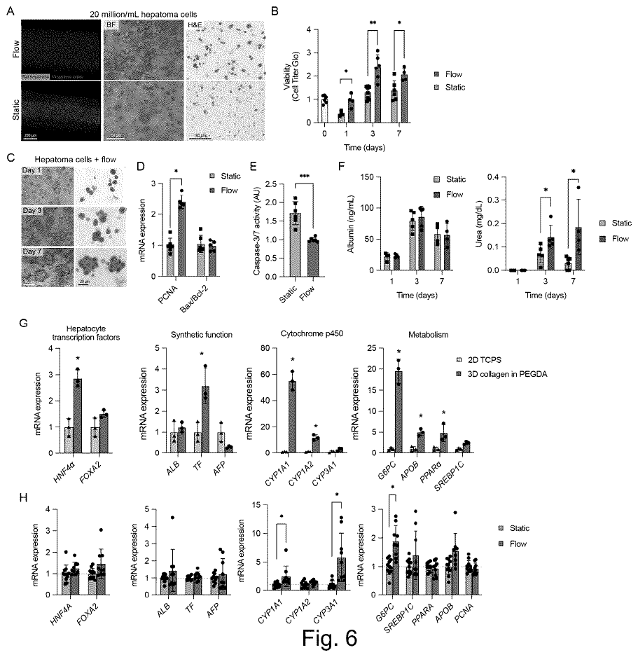

Under flow/perfusion conditions, medium was pumped through the PV channel at 1 mL/min whereas 3DPDs in static conditions were maintained submerged in medium. At the indicated times, cell/collagen matrices were removed from the HB channels for processing. Under static conditions, early time points revealed areas of cell death as indicated by propidium iodide, brightfield, and histological staining (FIG. 6A). Whole cell/collagen matrices were also lysed, and ATP was quantified as a measure of viability. At 24 hours after loading, the high initial cell density led to cell death in static conditions before recovery at 3 and 7 days (FIG. 6B). In contrast, perfused devices maintained cell viability and increased in cell density over 1 week (FIG. 6C). Further analysis of early time points showed a relative increase in expression of the proliferation marker gene PCNA for flow conditions. Although the mRNA expression ratios of pro-apoptosis to anti-apoptosis genes Bax/Bcl2 were unchanged, caspase-3/7 enzyme activity was 70% higher without flow (FIG. 6D-6E).

An important liver function is synthesis and secretion of proteins and compounds by the hepatocyte. Hepatocyte-specific synthetic products secreted into the surrounding hydrogel and medium were measured. Albumin levels, a sensitive marker of secreted proteins, were not affected by perfusion status, but urea production increased in the presence of flow (FIG. 6F). Next, gene expression in hepatoma cells cultured in 3DPDs was compared to that in standard tissue culture plastic where diffusion in medium is not limiting. RNA was extracted from cells grown for 7 days in 3DPDs and flasks and analyzed by RT-PCR. Cells cultured in 3DPDs had equivalent or substantially greater gene expression across core hepatocyte functional domains including hepatocyte-specific transcription factors, synthetic products, cytochrome p450s, and metabolic genes (FIG. 6G). In a comparison of gene expression from cells in 3DPDs with or without flow, the present disclosure found p450 genes CYP1A1 and CYP3A4 as well as glucose metabolism gene G6PC to be differentially induced (FIG. 6H).Thus, the present disclosure confirmed that perfusion in 3DPDs enhanced viability of rat hepatoma cells within devices and promoted gene expression in key hepatocyte-specific categories. Further, cells within 3DPDs exhibited greater gene expression in comparison to standard tissue culture conditions. This is likely due to the superior culture environment within a 3D collagen matrix in terms of substrate composition and stiffness in addition to 3D vs. 2D cell attachment.

### Primary Rat Hepatocytes Maintain Viability and Function in 3D Printed Devices.

In contrast to cell lines and some other primary cell types, primary hepatocytes are notoriously sensitive in vitro, poorly proliferative, and difficult to maintain for long periods of time. The present disclosure thus analyzed the behavior of primary rat hepatocytes cultured in 3DPDs. At low hepatocyte densities, total viability as measured by ATP was comparable at day 3 and day 7. At higher cell densities, introduction of flow better maintained cell viability (FIG. 7A), and albumin production scaled similarly to viability at these time points (FIG. 7B). The present disclosure also observed these results in cryopreserved primary rat hepatocytes (FIG. 7C). Similar to rat hepatoma cells, the expression of select p450 genes and G6PC were 2- to 6-fold higher with flow in primary hepatocytes (FIG. 7D). The present disclosure confirmed that flow-induced p450 gene expression led to an increase in enzyme activity using a CYP3A4 (human ortholog to rat CYP3A1) activity assay (FIG. 7E) and that perfusion of rifampicin through the PV channel could further induce CYP3A4 (FIG. 7F).

In this in vitro system with primary rat hepatocytes, the present disclosure also sought to determine whether perfusion of medium through the PV was sufficient to maintain cell viability. Specifically, in both static and flow setups, 3DPDs are submerged in medium available to cells via bulk diffusion through the hydrogel from the outside, potentially providing a source of glucose and soluble nutrients. However, if 3DPDs are implanted and anastomosed directly to the vasculature, the only significant source of soluble nutrients is the blood in the PV channel; the abdominal cavity likely provides little for bulk diffusion. The present disclosure therefore designed a perfusion setup in which medium perfuses through the PV but is not available in the environment surrounding the 3DPD and therefore cannot diffuse into the hydrogel via the 3DPD's external boundaries. This setup was compared to the default perfusion setup in which medium is available from both the outside and via the PV channel (FIG. 7G). This was also compared to a static setup in which only media contained in the bulk hydrogel is available to cells. The present disclosure found that cell viability was equivalent regardless of bulk hydrogel diffusion, provided there was flow in the PV channel. However, in absence of PV flow, bulk diffusion from medium outside the 3DPD was required to maintain some level of viability (FIG. 7H-7I). Therefore, the present disclosure found PV channel perfusion to be necessary and sufficient to maintain cell viability in the 3DPDs.

### Co-Cultures of Hepatocytes, Endothelial Cells, and Mesenchymal Stem Cells Demonstrate Features of Tissue Architecture and Function.

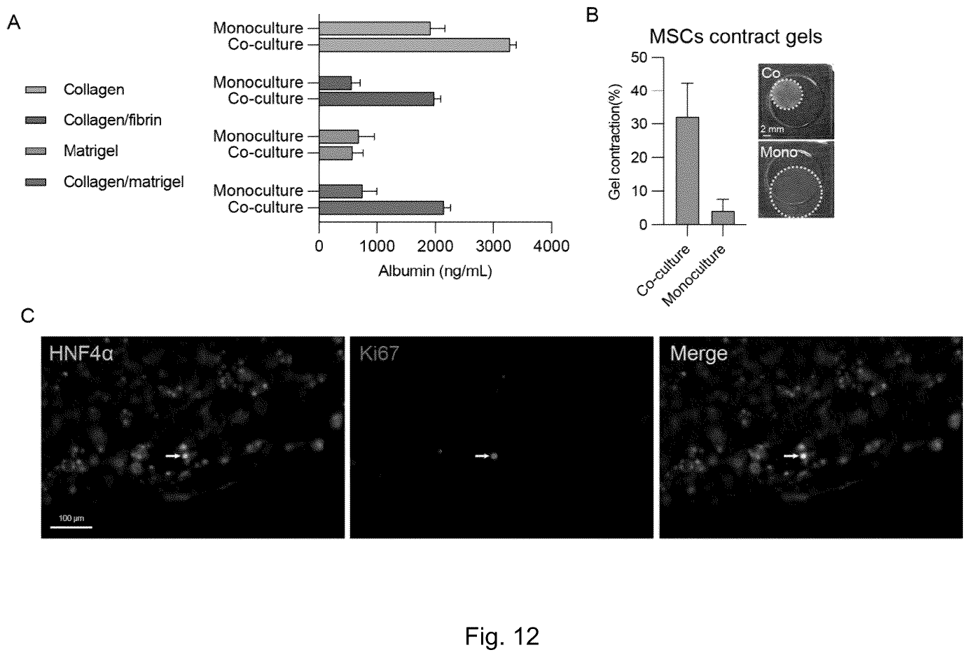

In evaluating co-cultures of primary rat hepatocytes, endothelial cells, and bone-marrow MSCs in 3D matrices, the present disclosure found that collagen alone supported the greatest albumin production in comparison to other naturally derived biomaterials (FIG. 12A). In contrast to monocultures of hepatocytes, the co-cultures—and MSCs in particular—assist with remodeling and contraction of the gel to produce a more compact 3D culture (FIG. 12B) as has been observed previously in co-cultures of iPSCs, HUVECs, and MSCs. Additional details and information is found at T. Takebe et al., Vascularized and functional human liver from an iPSC-derived organ bud transplant. Nature 499, 481-484 (2013), which is hereby incorporated by reference in its entirety for all purposes.

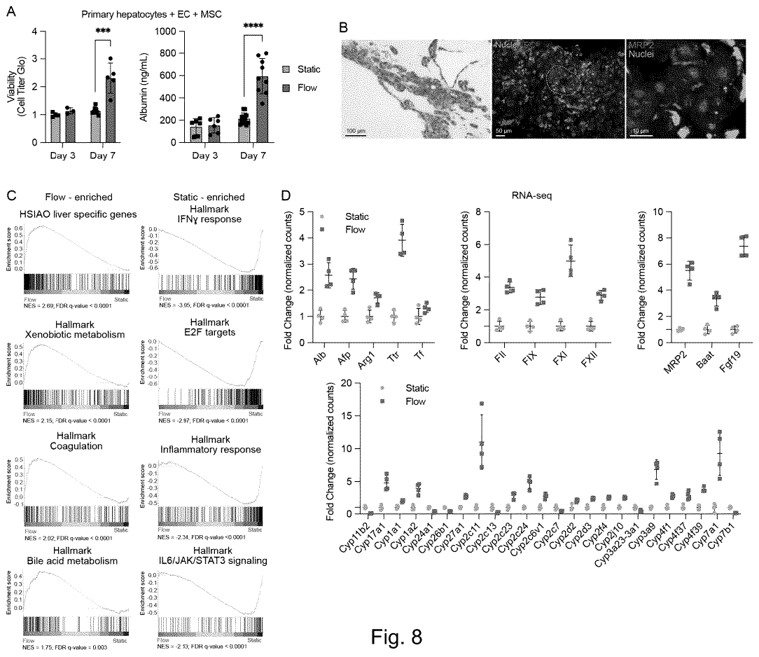

In 3DPDs, the present disclosure found that flow enhanced the total cell viability and albumin production at day 7 (FIG. 8A). The increase in cell number between day 3 and day 7 under flow is likely due to expansion of endothelial cells and MSCs, although some proliferating hepatocytes (HNF4α+/Ki67+) were noted at day 7 (FIG. 12C). Microscopy of the co-cultures in 3DPDs with perfusion revealed signs of liver tissue architecture such as hepatocyte rosettes (typical regenerative structures) and apical-basal polarization with localized MRP2 bile transporter expression (FIG. 8B).

The present disclosure then analyzed the effect of flow on gene expression of co-cultures in 3DPDs by subjecting day 7 3DPDs to bulk RNA-seq analysis. With flow, gene set enrichment analysis (GSEA) found broad enrichment of liver function-specific gene sets such as xenobiotic metabolism, coagulation, and bile acid metabolism. In contrast, without flow, cells within 3DPDs were enriched in upregulated genes related to cell cycle, inflammation, IL6/JAK/STAT, and interferon signaling (FIG. 8C). Individually selected genes relevant to these liver-specific categories show >2-fold increases in hepatocyte synthetic products such as albumin, clotting factors, and bile production. Expression of many p450s were differentially regulated; most were upregulated under flow (FIG. 8D).

### Devices can be Anastomosed In Vivo and Maintain Cell Viability.

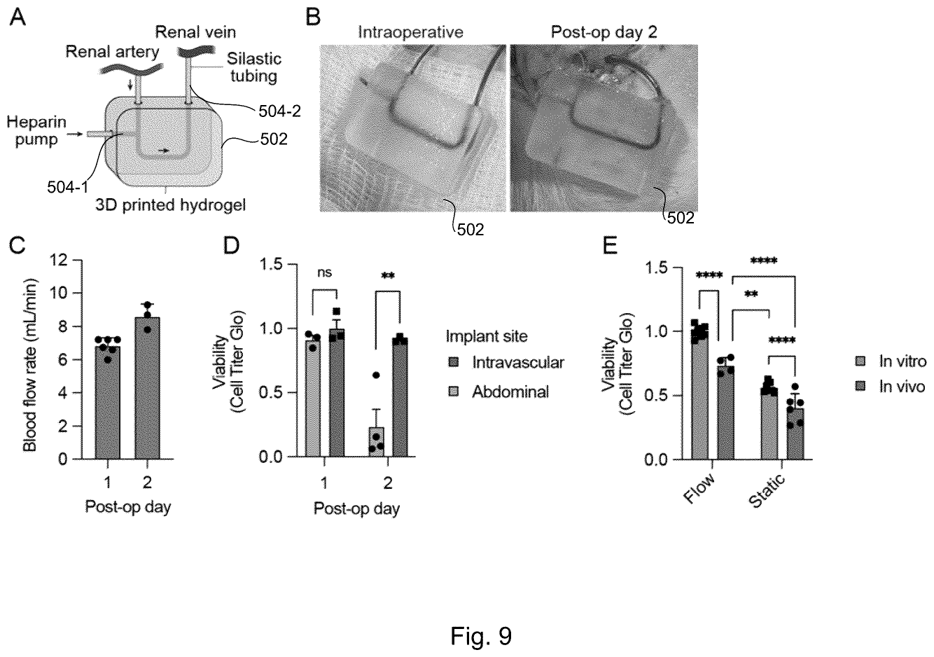

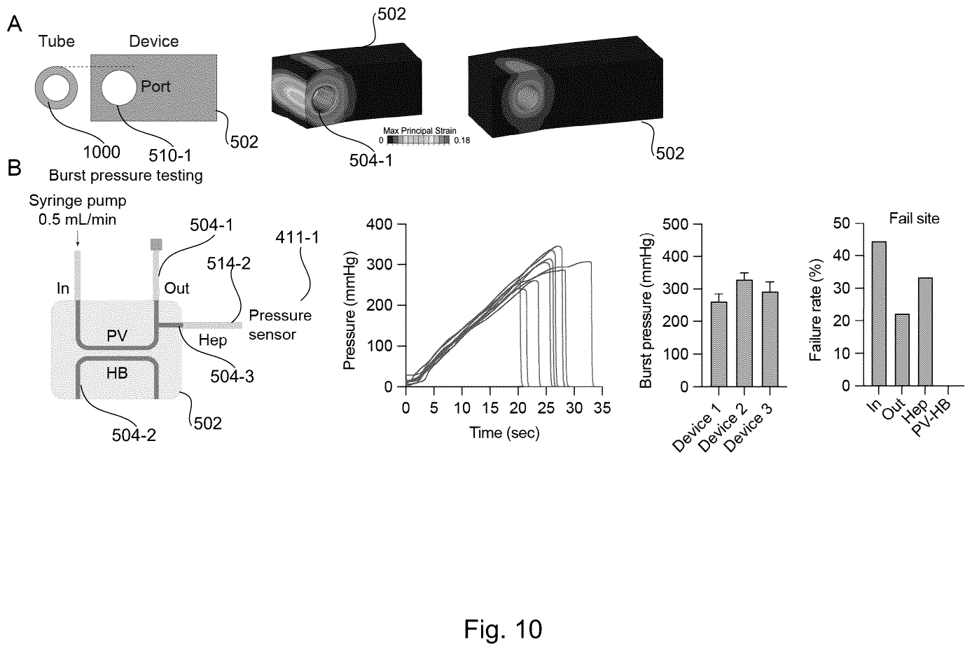

A surgical technique for implanting 3DPDs in a rat liver orthotopic model is provided. Additional details and information is found at T. Sahakyants et al., Rodent Model for Orthotopic Implantation of Engineered Liver Devices. Tissue Eng Part C Methods 29, 20-29 (2023), which is hereby incorporated by reference in its entirety for all purposes. The present disclosure modified this technique as a renal shunt in which the 3DPD is connected to the renal artery inflow and renal vein outflow (FIG. 9A). The microsurgery involved the cannulation of the left renal artery and vein using separate silastic tubing, each matching the diameter of the respective vessel. The free ends of these tubes were then connected to the inflow and outflow ports of the 3DPD, and blood flow through the 3DPD was established following the left nephrectomy. Critically, this procedure allows the implant to receive direct arterial blood flow through the PV channel, distinguishing it from other implantation methods such as a subcutaneous placement, which often relies on neovascular ingrowth to graft.

Although PEGDA resists protein adsorption and thrombogenesis, rats are known to be hypercoagulable and require additional heparinization. The in vivo 3DPD was thus modified to accommodate an additional port that provides heparin directly to the PV channel. An osmotic heparin pump was therefore implanted into the lumbar subcutaneous pocket and connected via silastic tubing to the additional port. This connection was facilitated through a surgically created tunnel between the lumbar subcutaneous pocket and the abdominal cavity where the 3DPD was positioned. This setup directly and continuously infused heparin into the blood-containing PV channel. Blood flow through the PV channel was rapidly established (<1 sec) after the silastic tubes were unclamped. Pulsations due to the arterial inflow were observed in the heparin inflow tubing. As both the 3DPD and the pump delivering heparin were internalized, this implementation generated a freely moving animal model suitable for long-term studies.

Effective anticoagulation and continuous blood flow was achieved 2 days post-operative as indicated by the bright red arterial blood within the PV channel (FIG. 9B). The square cross section of the channel minimizes surface irregularities that may potentially be conducive to initiating thrombosis in comparison to the 3D printed circular cross-sections (FIG. 19). The flow rate of blood at sacrifice post-operative day 1 was 6.83±0.31 mL/min and increased slightly at day 2 (FIG. 9C). Flow rate increases over time as the intraoperative vasospasm and the effects of anesthesia resolve. In total, 15 surgeries were successfully performed in which devices were assessed at post-operative day 1. Of those devices, one device later failed due to a dislodged outlet tube leading to intraabdominal bleeding. One device was clotted and no blood flow through the device was observed. Of the 6 additional devices successfully implanted and assessed at post-operative day 2, two were clotted. The present disclosure found that optimizing port dimensions was critical in preventing mechanical failures intraoperatively as well as post-recovery of the animal following anesthesia.

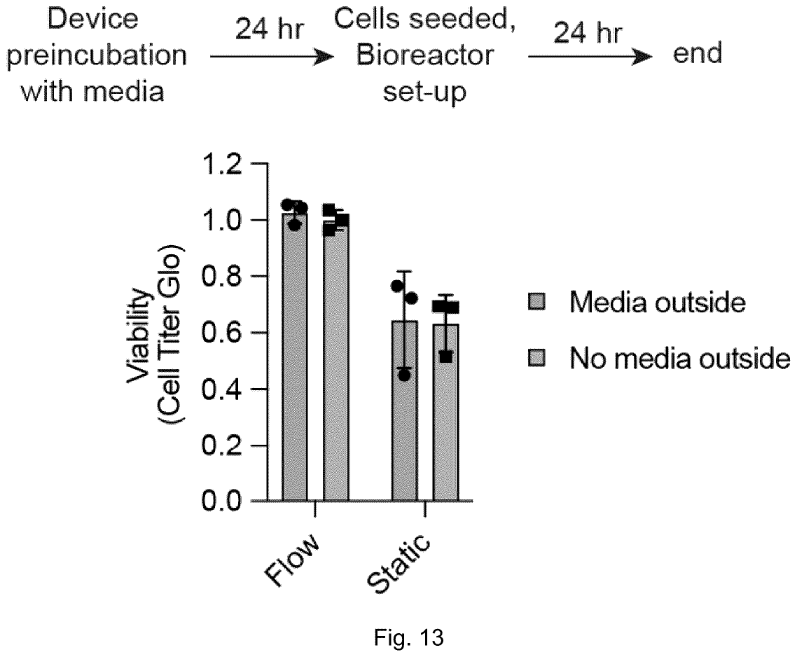

3DPDs were loaded with rat hepatoma cells and either implanted in place of the kidney intravascularly or placed in the abdomen without blood flow as analogs of flow and static conditions. The present disclosure found no difference in cell viability at post-operative day 1 between the intravascular and abdominally placed 3DPDs but a large increase in cell death was apparent at day 2 in abdominally placed 3DPDs (FIG. 9D). As rat hepatoma cells are hardier than primary hepatocytes, the present disclosure speculated that significant numbers of cells could survive at 24 hours; indeed, hepatoma cells in static devices without additional media maintained similar viability to static devices with additional media at 24 hours in vitro (FIG. 13).

Given primary hepatocytes' sensitivity to culture conditions, the present disclosure then assessed the viability of primary hepatocytes isolated from Wistar rats, loaded into 3DPDs, and implanted in vivo. Devices were cultured under flow bioreactor conditions for 3 days after which they were implanted in immunocompetent littermates either intravascularly or abdominally. As a control, two groups of devices remained in vitro with or without flow. At post-op day 1, devices implanted intravascularly contained 1.88-fold more viable hepatocytes compared to abdominally implanted devices. Although in vitro perfused devices contained more viable cells than in vivo intravascular implants, the viability of in vivo perfused devices was significantly greater than both in vitro and in vivo static groups (FIG. 9E). Thus, the present disclosure successfully developed an intravascular implantation of the 3DPDs and demonstrated that blood flow within the implant improved viability compared to non-perfused implants.

### Two-Channel Design can be Scaled Using Fractal Geometry.

To demonstrate that the two-channel design motif can be scaled to accommodate larger cell mass, the present disclosure employed a fractal geometry to fill 3D space while preserving the proximity of PV channels to HB channels. The present disclosure hypothesized that cell viability could be maintained in 3DPDs while increasing total cell number due to the perfusability of PV channels.

The fractal-like channel network geometry consists of one or more generations of T-junctions where a parent channel of diameter D₀ bifurcates at right angles into two child channels with diameters 21/3D₀, the scaling from Murray's law. Higher channel complexity is achieved both by increasing the number of generations within a layer and creating stacks of layers (FIG. 20A-20B). These layers are fed by a bifurcating distributor channel network and drained by a bifurcating collector channel network, thereby connecting each fractal architecture into a single system. To ensure uniform flow distribution across a layer, the T-junctions at each generation are identical and channel segments connecting T-junctions between two given generations are also identical. To ensure that each layer in the stack is fed the same flow rate, the present disclosure chose the distributor and collector networks to be identical, as well as the channel segments connecting the distributor/collector networks to layers. Taken together, this design strategy ensures that any flow path through the device has the same length and flow resistance, at least in the low Reynolds number limit.

The present disclosure designed a scaled 3DPD with 4 generations in which the smallest channel diameter was 400 μm (FIG. 14A-14F). The nominal volume of HB channels containing hepatocytes increased by about 7.7-fold compared to the non-bifurcated original two-channel design with an overall device width of 3 cm. These networks were interfaced with the inlet, outlet, and heparin ports.

A larger bioreactor container was designed to support in vitro perfusion of the PV network with a peristaltic pump (FIG. 20C). With 20 million/mL rat hepatoma cells, the present disclosure verified that perfusion is essential to sustaining this cell density over time (FIG. 14G-14H). Perfusion of the scaled 3DPD containing primary hepatocytes also significantly increased viability compared to static devices (FIG. 20D). The present disclosure then implanted empty scaled 3DPD devices in the renal shunt rodent model. After unclamping cannula tubing to permit blood flow, all PV channels filled with blood (FIG. 14I). Patency to blood flow was maintained at post-operative day 1 with an average flow rate of 4.8 mL/min (N=2).

Here, the present disclosure describes a general strategy for the application of 3D printed vascular-like structures for direct implantation of cell-laden scaffolds. Small molecules diffuse across a permeable hydrogel barrier separating separate vascular and cell compartments to maintain higher cell counts and increased functionality. Empirical testing of the mass transport properties was conducted in vitro with dye diffusion and with viability of rat hepatocytes in vitro and in vivo. The design of physical connections between tubing and 3DPDs was optimized to provide adequate hydrogel mechanical integrity, bioreactor perfusion, and in vivo implantation.

The inclusion of flow as a cell culture parameter may also benefit hepatocyte function beyond viability alone. In rat hepatoma, primary rat hepatocyte, and co-culture loaded 3DPDs, gene expression and p450 activity were highly induced. Induction of p450 activity may be due to higher oxygen tension in the media or the effects of low shear stress, and in hepatocyte culture setups in which medium flow acts directly on monolayers, induction of p450 activity has been observed in numerous studies. Additional details and information is found at S. Kidambi et al., Oxygen-mediated enhancement of primary hepatocyte metabolism, functional polarization, gene expression, and drug clearance. Proceedings of the National Academy of Sciences of the United States of America 106, 15714-15719 (2009); B. Vinci et al., Modular bioreactor for primary human hepatocyte culture: medium flow stimulates expression and activity of detoxification genes. Biotechnol J 6, 554-564 (2011); I. Shvartsman, T. Dvir, T. Harel-Adar, S. Cohen, Perfusion cell seeding and cultivation induce the assembly of thick and functional hepatocellular tissue-like construct. Tissue Eng Part A 15, 751-760 (2009); P. Roy, J. Washizu, A. W. Tilles, M. L. Yarmush, M. Toner, Effect of flow on the detoxification function of rat hepatocytes in a bioartificial liver reactor. Cell Transplant 10, 609-614 (2001); E. Novik, T. J. Maguire, P. Chao, K. C. Cheng, M. L. Yarmush, A microfluidic hepatic coculture platform for cell-based drug metabolism studies. Biochem Pharmacol 79, 1036-1044 (2010), each of which is hereby incorporated by reference in its entirety for all purposes. Similarly, perfusion status of hepatic organoids in PEGDA microfluidic arrays modulates phenotype, including p450 and gluconeogenesis (G6PC). Additional details and information is found at S. Grebenyuk et al., Large-scale perfused tissues via synthetic 3D soft microfluidics. Nature communications 14, 193 (2023), which is hereby incorporated by reference in its entirety for all purposes. Importantly, hepatocytes are very mechanically sensitive and are not subjected to significant tangential shear flow in tissue; high flow rates in vitro leading to high shear stress can negatively affect hepatocyte function. Additional details and information is found at A. W. Tilles, H. Baskaran, P. Roy, M. L. Yarmush, M. Toner, Effects of oxygenation and flow on the viability and function of rat hepatocytes cocultured in a microchannel flat-plate bioreactor. Biotechnol Bioeng 73, 379-389 (2001), which is hereby incorporated by reference in its entirety for all purposes. In some embodiments, the 3DPD channel design of the present disclosure includes 5 mL/min blood flow induces about 20 dyne/cm2 of wall shear stress (FIG. 21), which is within the normal physiological range of human arteries. Importantly however, the hepatocytes are protected from direct shear stress by the hydrogel barrier, similar to the buffer formed by the space of Disse in vivo.

The present disclosure chose PEGDA as a suitable proof-of-concept printing material due to properties that were compatible with printability and surgical implantation. As a class of biomaterials, hydrogels often suffer from poor mechanical stability. PEGDA, although more robust than many other hydrogel polymers, can be brittle, making surgical implantation and integration with other materials such as tubing challenging. However, the present disclosure found that the combination of optimized photoink formulation, printing settings, and mechanical testing supported many successful implantations in rodents that were often very active post-operatively. PEGDA is a popular and easily produced polymer, and a PEGDA photoink can be produced economically which may be critical to the affordability of large 3D printed structures. More generally, other 3D printing techniques may allow printing multiple materials which could better address mechanical stability and permeability. Additionally, other photoink polymer blends can exhibit advantageous tough or strain-tolerant mechanical behavior. Additional details and information is found at Y. T. Kim, A. Ahmadianyazdi, A. Folch, A ‘print-pause-print’ protocol for 3D printing microfluidics using multi-material stereolithography. Nat Protoc 18, 1243-1259 (2023); J. Y. Sun et al., Highly stretchable and tough hydrogels. Nature 489, 133-136 (2012); Ge et al., 3D printing of highly stretchable hydrogel with diverse UV curable polymers. Sci Adv 7 (2021), each of which is hereby incorporated by reference in its entirety for all purposes.

The choice of PEGDA was also intended to reduce protein and platelet adsorption leading to thrombogenesis. The present disclosure observed occasional clotting of 3DPDs in vivo, and this was likely related to the accessed vascular site and resulting blood flow rate. Chemical modifications of PEGDA, such as inclusion of RGD peptides, or replacement with related polymers such as GelMA, may promote the cell-material interactions to endothelialize channel walls for additional protection against thrombogenesis. Biodegradable polymers such as GelMA or ColMA can also be employed to promote neovascular ingrowth into the hydrogel which may further enhance inter-channel transport. However, the lack of bioactivity of the PEGDA hydrogel in this work was offset by filling channels with a collagen hydrogel as a cell carrier. Other biomaterial matrices may offer greater benefits to the development of tissue-like architecture; for example, inclusion of fibrin is often used to enhance endothelial tubule formation, and optimization of matrix stiffness may be important to coordinating liver tissue development and function. Additional details and information is found at D. B. Kolesky, K. A. Homan, M. A. Skylar-Scott, J. A. Lewis, Three-dimensional bioprinting of thick vascularized tissues. Proceedings of the National Academy of Sciences of the United States of America 113, 3179-3184 (2016); T. A. Ahmed, E. V. Dare, M. Hincke, Fibrin: a versatile scaffold for tissue engineering applications. Tissue Eng Part B Rev 14, 199-215 (2008); G. Sorrentino et al., Mechano-modulatory synthetic niches for liver organoid derivation. Nature communications 11, 3416 (2020), each of which is hereby incorporated by reference in its entirety for all purposes.

Direct intravascular implantation of 3D printed or microfabricated structures resembling microfluidic devices is not often attempted due to technical difficulties such as thrombogenesis. In previous work, the present disclosure developed microfluidic arrays and implanted them with direct blood flow from the femoral artery in rats. Additional details and information is found at W. M. Hsu et al., Liver-assist device with a microfluidics-based vascular bed in an animal model. Ann Surg 252, 351-357 (2010), which is hereby incorporated by reference in its entirety for all purposes. Channels remained patent and perfused for 24 hours, but only with regular manual bolus heparin injections. Here, the present disclosure introduced continuous heparin delivery through osmotic pumps, which were easily integrated into 3DPD channels and enabled the 3DPDs to be implanted in the renal artery up to 2 days. Polydimethylsiloxane (PDMS) microfluidic devices have also been implanted in the femoral artery for up to 3 hours. Additional details and information is found at R. Sooppan et al., In Vivo Anastomosis and Perfusion of a Three-Dimensionally-Printed Construct Containing Microchannel Networks. Tissue Eng Part C Methods 22, 1-7, (2016), which is hereby incorporated by reference in its entirety for all purposes. GelMA/fibrin gels have been implanted in the carotid artery for 1 week, albeit with thrombus formation. Additional details and information is found at X. Liu et al., 3D Liver Tissue Model with Branched Vascular Networks by Multimaterial Bioprinting. Adv Healthc Mater 10, e2101405 (2021), which is hereby incorporated by reference in its entirety for all purposes. Both Zhang et al and Szklanny et al deployed endothelialized degradable polymers in a rodent femoral artery model over 1 and 2 weeks. Additional details and information is found at B. Zhang et al., Biodegradable scaffold with built-in vasculature for organ-on-a-chip engineering and direct surgical anastomosis. Nat Mater 15, 669-678 (2016); A. A. Szklanny et al., 3D Bioprinting of Engineered Tissue Flaps with Hierarchical Vessel Networks (VesselNet) for Direct Host-To-Implant Perfusion. Adv Mater 33, e2102661 (2021), each of which is hereby incorporated by reference in its entirety for all purposes. 3D printed PEGDA was also used as an arteriovenous conduit in a large animal model in which different anatomical implantation sites were necessary to prevent clotting. Additional details and information is found at N. T. N. Galvan et al., Blood Flow Within Bioengineered 3D Printed Vascular Constructs Using the Porcine Model. Front Cardiovasc Med 8, 629313 (2021), which is hereby incorporated by reference in its entirety for all purposes. Other directly implanted tissue engineered scaffolds, such as decellularized organs, similarly have susceptibility to clotting without endothelial linings. Additional details and information is found at B. E. Uygun et al., Organ reengineering through development of a transplantable recellularized liver graft using decellularized liver matrix. Nat Med 16, 814-820 (2010); J. Devalliere, Y. Chen, K. Dooley, M. L. Yarmush, B. E. Uygun, Improving functional re-endothelialization of acellular liver scaffold using REDV cell-binding domain. Acta Biomater 78, 151-164 (2018), each of which is hereby incorporated by reference in its entirety for all purposes.

The present disclosure observed that intravascular perfusion of 3DPDs was critical for increasing cell viability in comparison to abdominally placed devices not connected to blood flow. Currently however, even with perfusion, cell death was increased compared to in vitro devices at matched time points. Several factors may contribute to this phenomenon, including reperfusion injury resulting in inflammatory cytokines and the use of allogenic cells in an immunocompetent rodent model. In on-going work, the present disclosure are developing protocols for use of autologous primary and stem cells, as well as appropriate surgical management.

The present disclosure also note that this current design, in which parenchymal cells are included within a channel, constitutes a second fluidic network in addition to the blood-including portal-venous channel. In an ideal application in a surgical model, HB terminal ports could be connected via tubing to an existing bile network, thus allowing bile produced by loaded cells to drain into the biliary system.

A second goal of the present disclosure focuses on iterating and scaling the parallel channel motif to increase channel volumes and therefore the cell mass deliverable in an implant. Whereas reliance on diffusion by non-perfusable or avascular constructs limits scaling, the present disclosure hypothesize that the inclusion of sufficiently dense channels can sustain tissue deep within large implants. Indeed, the present disclosure observed that fractally scaled 3DPDs demonstrate an increased dependence on flow for cell survival in comparison to the smaller two-channel design. Suitable surgical support systems are currently being developed to evaluate these larger scaled implants in a large animal model.

### Materials and Methods

### Experimental Setup.

The design for a bi-channel 3D device to support liver cell survival and function was constructed in SolidWorks and synthesized of poly(ethylene glycol) diacrylate with a direct light 3D printer. One channel (HB) was designed to be seeded with liver cells suspended in a hydrogel. The second channel (PV) supports the cells with medium or blood. Active laminar flow brings fresh soluble factors to and removes soluble waste from the PV side of the PV-HB membrane. The fresh soluble factors then diffuse through the membrane to reach cells in the HB channel, and vice versa for the waste factors. The device is designed with ports to readily allow anastomosis to a peristaltic pump or host vasculature upon implantation.

Multiple parameters potentially influencing hepatocyte survival and function were evaluated during optimization. These variables included cell density, biomatrix composition, inclusion of nonparenchymal cell types, and the flow of medium and blood. Evaluation included both general cell physiology and liver-specific functions.

### 3D Printing.

Computer-aided design (CAD) files of 3D printed devices were constructed in Solidworks and printed on a LUMEN-X DLP printer using 100 m layer resolution polymerized at 405 nm. The photoink formulation was 20% poly(ethylene glycol) diacrylate (PEGDA) molecular weight 700 Da, 0.5 wt % lithium phenyl-2,4,6-trimethylbenzoylphosphinate (LAP), 2 mM tartrazine, and 10% 10×PBS. Each layer was UV polymerized for 3.5 sec for a total print time of −15 min. After printing, completed parts were sterilized in 70% ethanol for 24 hours and washed extensively with large volumes of PBS.

### Cell Culture and Isolation.

H4-II-E-C3 rat hepatoma cells were obtained from ATCC and cultured in high glucose DMEM, 20% horse serum, 5% fetal bovine serum, and 1% penicillin/streptomycin. Rat liver endothelial cells were obtained from Cell Biologics and cultured in endothelial growth medium (EGM-2, Promocell). Rat bone marrow mesenchymal stem cells (BM-MSCs) were either obtained from Cell Biologics or isolated in-house from rat femurs and tibias according to established protocols. Additional details and information is found at M. Soleimani, S. Nadri, A protocol for isolation and culture of mesenchymal stem cells from mouse bone marrow. Nat Protoc 4, 102-106 (2009), which is hereby incorporated by refence in its entirety for all purposes. BM-MSCs were cultured in StemXVivo.

Primary rat hepatocytes were isolated according to previous protocols. Additional details and information is found at P. O. Seglen, Preparation of isolated rat liver cells. Methods Cell Biol 13, 29-83 (1976), which is hereby incorporated by reference in its entirety for all purposes. Briefly, 2-3 month old Wistar rats were used in accordance with the Charles River Accelerator and Development Lab IACUC. Animals were anesthetized with isoflurane and intravascularly injected with a bolus of heparin. The portal vein was cannulated and the liver flushed with lactated Ringer's solution including mannitol and heparin. After transport on ice, livers continued perfusion with cation-free Hank's Balanced Salt Solution (HBSS) with ethylene glycol-bis(3-aminoethyl ether)-N,N,N′,N′-tetraacetic acid, followed by cation-including HBSS, and a 0.05% collagenase IV (Sigma C5138) digestion buffer. Following digestion, livers were minced with scissors and washed with HBSS at 50×g. The hepatocyte fraction was further purified in a 50% Percoll gradient. Isolations generally yielded >90% viability.

### Device Loading and Bioreactor Setup.

Cells were introduced to the hepatobiliary compartment of the 3D printed device by suspending the cells in a solution of rat tail collagen and pipetting the mix into the channel. A 3 mg/mL collagen solution was prepared by mixing high concentration rat tail collagen (Advanced Biomatrix, 10 mg/mL), 5×DMEM, 7.5% sodium bicarbonate, and 100 mM HEPES. For H4-II-E-C3 rat hepatoma experiments, the final cell density in the collagen matrix was 20 million/mL. For primary rat hepatocyte monocultures, a density of 4 million cells/mL was used unless otherwise indicated. For co-culture experiments, 1 million hepatocytes/mL were mixed with endothelial cells and mesenchymal stem cells at a 4:2:1 ratio.

Devices were incubated at 37° C. for 1 hour to allow the collagen to set. To prevent abdominal blood from entering the cell compartment channels for in vivo implantations, the inlet and outlet ports were plugged with silastic tubing filled with silicone glue.

In vitro bioreactor chambers were constructed using 50 mL conical tubes. Holes were drilled in the cap of a tube to introduce inlet and outlet tubing. A third hole was created to introduce a sterile vent filter capsule. Approximately 250 cm of platinum-cured silicone tubing (ID 1/32″, OD 1/16″) was connected to white/white stop tubing (PharMed BPT) clipped into a Harvard Apparatus P-70 peristaltic pump. The silicone tubing was connected to 3D printed devices and 30 mL of media was pumped through the portal-venous channel. Unless otherwise indicated, a flow rate of 1 mL/min was established. For bioreactor setups that included no external medium, a separate tube 8 including 30 mL of medium was used to recirculate the medium within the device channels. To maintain a humidified environment, gauze soaked with PBS was placed at the bottom of the conical tube. 3DPDs in flow or static conditions were suspended above soaked gauze to prevent contact between the gauze and 3DPD hydrogel.

For all bioreactor experiments, serum-free hepatocyte growth media (HGM, Lonza) was used. In co-culture experiments, HGM was mixed 1:1 with EGM-2.

### Device Characterization.

Acellular hydrogel devices were cut with a razor blade to expose internal channels and prepared for scanning electron microscopy by an ethanol dehydration series followed by hexamethyldisilazane and vacuum desiccation. Hydrogel pieces were sputter coated with Au/Pd and imaged on a Zeiss Gemini Sigma 300 VP FE-SEM.

To determine burst pressure of tube-hydrogel connection, a syringe pump (Harvard Apparatus) injected PBS through the inlet tubing at 0.5 mL/min. The outlet tubing was clamped and the heparin port tubing terminated at a digital pressure sensor. As the syringe pump generated pressure within the device, pressure measurements were recorded.

The ability of small molecules to diffuse through the hydrogel barrier between channels was assessed by perfusing a rhodamine solution through the PV channel with a TTP Ventus positive pressure displacement pump. At indicated time points, fluid was collected from the HB channel and rhodamine concentration was determined by reading fluorescence at 526/550 ex/em.

Mechanical characterization of PEGDA hydrogel was conducted using 1.5 cm cubes for compression tests and dogbone geometries for tensile tests. The data was obtained on a TA Instruments Discovery HR 20 Hybrid Rheometer using tension and compression geometries and analyzed with TRIOS software. Tension measurements were performed at a constant linear rate of 20 m/s; compression measurements were performed at a constant linear rate of 10 m/s.

Hydrogel swelling parameters were obtained by 3D printing cylinders (1.5 cm diameter, 1 cm height). Gels were weighed or measured for volume before and after swelling in water or PBS. Eluted photomask (tartrazine) was measured by absorbance at 430 nm and quantified with a standard curve.

### Surgical Implants.

All animal experiments were reviewed and approved by the Institutional Animal Care and Use Committee (IACUC). Male Wistar rats (Crl:WI Wistar rats; RRID:RGD_737929; Charles River Laboratories, Wilmington, MA) weighing 350-450 g were habituated for a minimum of 3 days before use in experiments. Animals were maintained on a 12-h light/12-h dark cycle and given rat chow and water ad libitum. Rats were housed in pairs before surgery and individually after surgery to enable undisturbed recovery.

A procedure for anastomosing 3D printed hydrogel devices as a liver orthotopic implant in a rat is provided. Additional details and information is found at T. Sahakyants et al., Rodent Model for Orthotopic Implantation of Engineered Liver Devices. Tissue Eng Part C Methods 29, 20-29 (2023), which is hereby incorporated by reference in its entirety for all purposes. Here, the present disclosure adapted this surgical technique to supply the device with blood originating from the renal artery and draining into the renal vein. The animals were initially anesthetized using isoflurane, with induction at 4% and maintenance between 1.5-2%, supplemented with an oxygen flow of 1 L/min, administered via a nose cone connected to an anesthesia machine. Subsequently, a subcutaneous pocket was created in the lumbar area, adequately sized to accommodate an Alzet 2ML1 pump (Durect #0000323), which was pre-filled and primed with a heparin sodium solution (1000 USP units/mL, Sagent Pharmaceuticals #25021-400-10). A transverse incision, measuring 3-4 cm, was then executed in the left lower abdominal quadrant to reveal the left kidney along with its vasculature. This was followed by the creation of a 7-8 cm tunnel traversing the left lateral lumbar region, designed specifically to house a silicone tube linking the Alzet pump to the abdominal cavity. Under the magnification provided by a Leica S9i stereomicroscope (Leica Microsystems #10450816), employing microsurgical techniques, the left renal artery and vein were meticulously dissected and isolated through blunt dissection. Cannulation was performed using platinum-cured silicone tubing, with inner diameters of 0.51 mm and 0.76 mm for the renal artery and vein, respectively. The left ureter was securely ligated. The left kidney was subsequently bluntly dissected, externalized, and excised. Within the abdominal cavity, two cell-loaded 3DPDs were placed; one was connected to arterial inflow, venous outflow, and heparin silicone tubes, receiving blood flow through the PV channel, while the other was positioned intraabdominally without blood flow. At postoperative day 1 and 2, blood flow rate was determined by cutting the outlet tubing and collecting the outflow immediately prior to euthanasia.

### Luminescent Assays.

Following indicated time points, the cell/collagen construct was manually removed from channels. The CellTiter-Glo 3D Cell Viability Assay (Promega) was used to determine cell viability and Caspase-Glo 3/7 Assay System (Promega) for apoptotic activity, with some modifications. For ATP-based viability determination, cell/collagen constructs were lysed in 10% trichloroacetic acid for 20 minutes on a plate shaker. For caspase 3/7 based apoptosis determination, cell/collagen constructs were lysed in Caspase Cell Lysis Buffer (Enzo Life Sciences). Lysate supernatants were then added to 50% diluted CellTiter Glo or Caspase-Glo reagents. Luminescence was read with a SpectraMax M5 plate reader at 0.5 s integration.

The p450-Gbo CYP3A4 activity assay (Promega) was used according to manufacturer's instructions with modifications. Where indicated, p450 activity was induced with 25 M rifampicin for 24 hours. Cell/collagen constructs were removed from channels and incubated with luciferase substrate for 1 hour. One volume of lytic detection reagent was added to wells and incubated for 20 min on a shaker. Lysates were read at 0.5 s integration.

### Viability Determination.

Nonviable cells were identified by propidium iodide staining and fluorescent imaging. In some cases, total cells were determined by staining with CellTracker Deep Red prior to cell loading or with end point calcein AM and Hoechst 33342. Images were acquired on a Zeiss 200 epifluorescent microscope.

### Albumin ELISA and Urea Assay.

Following indicated time points, conditioned media from bioreactors was collected. To ensure quantification of total albumin and urea production, the 3D printed hydrogel device was crushed and any hydrogel-trapped albumin/urea were allowed to elute into the media overnight at 4° C. Supernatants were centrifuged to remove debris and used in a rat albumin ELISA kit (Immunology Consultants Laboratory) or QuantiChrom Urea Assay Kit (Bioassay Systems).

### RT-qPCR and RNA-seq.

RNA was extracted from cells using Trizol and purified using the RNeasy Plus Universal Mini Kit (Qiagen). cDNA was synthesized using the High-Capacity cDNA Reverse Transcription Kit (Applied Biosystems) with 100 ng input RNA. qPCR was run on a StepOne Plus (Applied Biosystems) using SYBR green reagent. Gene expression differences were calculated using the 2-ΔΔCt method with 18S housekeeping gene.

RNA-seq library preparation with polyA selection, sequencing (Illumina HiSeq 2×150 bp), and mapping was performed by Azenta Life Sciences. Sequence reads were trimmed to remove possible adapter sequences and nucleotides with poor quality using Trimmomatic v.0.36. The trimmed reads were mapped to the Rattus norvegicus Rnor6.0 reference genome available on ENSEMBL using the STAR aligner v.2.5.2b. Relative gene expression analysis and gene set enrichment analysis (GSEA) were determined using the DEseq2 and GSEA module of GenePattern. Additional details and information is found at M. Reich et al., GenePattern 2.0. Nat Genet 38, 500-501 (2006), which is hereby incorporated by reference in its entirety for all purposes. For GSEA, normalized counts were compared against the hallmark and C2 gene sets in the Human Molecular Signatures Database.

### Histology and Staining.

Cell/collagen constructs were fixed in 4% PFA and embedded in either paraffin or OCT. Paraffin sections were used for hematoxylin and eosin staining. Cryosections were used for immunofluorescence. The following antibodies were used: MRP2 (Santa Cruz sc-59611), HNF4α (ThermoFisher MA1-199), and Ki67 (Abcam ab16667).

### Statistical Analysis.

The data were expressed as mean±SD and plotted in GraphPad Prism 10. Where present, each data point in a bar/scatter dot plot represents an individual 3DPD. Assays with arbitrary unit readouts (e.g. luminescent assays) were normalized such that “flow” or “static” experimental groups had a value of 1 such that experimental groups are expressed as fold-changes. FIGS. 3H and 5E were analyzed by a 2-way ANOVA with multiple comparisons correction using the Holm-Sidik method. FIGS. 2B, 2D, 2F, 2G, 2H, 3A, 3B, 3D, 4A, 5D, and 6G were analyzed by multiple unpaired t-tests with the Holm-Sidik correction. FIGS. 2E, 3C, 3E, and 3F were analyzed by unpaired t-tests. A P-value <0.05 was taken to be statistically significant. The following notations were used: ****P<0.0001, ***P<0.001, **P<0.01, *P<0.05.

### Exemplary Implementations

Various examples of aspects of the disclosure are described as numbered implementations (1, 2, 3, etc.) for convenience. These are provided a examples, and do not limit the subject technology.
Implementation 1. An implantable device comprising: a body made of a hydrogel material; a first intrinsic channel network formed in the body and configured to receive cells; and a second intrinsic channel network formed in the body and configured to allow for active flow of a fluid to support viability and/or function of the cells in the first intrinsic channel network, wherein the first intrinsic channel network comprises a first channel, the second intrinsic channel network comprises a second channel, at least a segment of the first channel and a segment of the second channel are parallel to each other and spaced apart from each other at a first distance, and the body between the segment of the first channel and the segment of the second channel forms a first nanoporous hydrogel barrier that allows for fluidic communication between the first channel and the second channel to nourish the cells.
Implementation 2. The implantable device of Implementation 1, wherein the device is made by 3D printing.
Implementation 3. The implantable device of any preceding Implementation, wherein the hydrogel material comprises polyethylene(glycol) diacrylate (PEGDA).
Implementation 4. The implantable device of Implementation 3, wherein the PEGDA has a molecular weight of about 700 Da.
Implementation 5. The implantable device of Implementation 3, wherein the PEGDA is chemically modified.
Implementation 6. The implantable device of any preceding Implementation, wherein the hydrogel material comprises 20% PEGDA, 0.5 wt % lithium phenyl-2,4,6-trimethylbenzoylphosphinate (LAP), 2 mM tartrazine, and 10% 10× polybutylene succinate (PBS).
Implementation 7. The implantable device of any one of Implementations 1-5, wherein the hydrogel material comprises GelMA or ColMA.
Implementation 8. The implantable device of any preceding Implementation, wherein the cells comprise parenchymal or hepatic cells.
Implementation 9. The implantable device of any preceding Implementation, wherein the cells are suspended in a collagen matrix.
Implementation 10. The implantable device of Implementation 9, wherein the collagen matrix comprises a collagen hydrogel.
Implementation 11. The implantable device of any preceding Implementation, further comprising: a plurality of ports formed at a boundary of the body and in fluidic communication with the first or second intrinsic channel network, wherein each respective port in the plurality of ports is configured to couple with a corresponding tube in a plurality of tubes, thereby allowing the implantable device to anastomose with a pump or host vasculature upon implantation.
Implementation 12. The implantable device of Implementation 11, wherein the corresponding tube is a silastic tube.
Implementation 13. The implantable device of any one of Implementations 11-12, wherein the plurality of ports is configured to keep a maximum principal strain of the body at a minimal level to prevent the body from cracking.
Implementation 14. The implantable device of any one of Implementations 11-13, wherein the plurality of ports comprises: a first port in fluidic communication with a first end of the second intrinsic channel network and configured to couple with a first tube in the plurality of tubes to receive the fluid for the second intrinsic channel network; and a second port in fluidic communication with a second end of the second intrinsic channel network and configured to couple with a second tube in the plurality of tubes to remove the fluid from the second intrinsic channel network.
Implementation 15. The implantable device of Implementation 14, wherein the first and second ports are configured such that connection between the first port and the first tube and connection between the second port and the second tube are stable in excess of a systolic pressure of about 300 mmHg in absence of an adhesive.
Implementation 16. The implantable device of any one of Implementations 11-151, wherein the plurality of ports further comprises a third port in fluidic communication with the first intrinsic channel network to drain a product produced by the cells.
Implementation 17. The implantable device of any one of Implementations 11-16, wherein the plurality of ports further comprises a fourth port in fluidic communication with the second intrinsic channel network to assist in flowing the fluid in the second intrinsic channel network.
Implementation 18. The implantable device of any one of Implementations 11-17, wherein the pump is peristaltic or osmotic.
Implementation 19. The implantable device of any preceding Implementation, wherein: the first intrinsic channel network comprises one or more first fractal architectures, each comprising one or more generations of first channel T-junctions; the second intrinsic channel network comprises one or more second fractal architectures, each comprising one or more generations of second channel T-junctions; and each respective first fractal architecture in the one or more first fractal architectures is adjacent to a corresponding second fractal architecture in the one or more second fractal architectures, thereby collectively forming a corresponding channel layer in one or more channel layers.
Implementation 20. The implantable device of Implementation 19, wherein the one or more channel layers are fed by a bifurcating distributor channel network and drained by a bifurcating collector channel network, thereby connecting each of the one or more first fractal architectures and each of the one or more second fractal architectures into a single system.
Implementation 21. The implantable device of any one of Implementations 19-20, wherein each of the one or more first fractal architectures comprises a plurality of generations of first channel T-junctions, and each of one or more second fractal architectures comprises a plurality of generations of second channel T-junctions.
Implementation 22. The implantable device of Implementation 21, wherein: first channel T-junctions at each generation are identical; channel segments connecting first channel T-junctions between two generations are identical; second channel T-junctions at each generation are identical; and channel segments connecting second channel T-junctions between two generations are identical.
Implementation 23. The implantable device of any one of Implementations 19-22, wherein the first intrinsic channel network comprises a plurality of first fractal architectures and the second intrinsic channel network comprises a plurality of second fractal architectures, thereby forming a plurality of channel layers stacked together.
Implementation 24. The implantable device of Implementation 23, wherein first fractal architectures in the plurality of first fractal architectures are identical to each other, and second fractal architectures in the plurality of second fractal architectures are identical to each other.
Implementation 25. An implantable device comprising: a body; a first intrinsic channel network formed in the body, wherein cells suspended in a collagen hydrogel are disposed in the first intrinsic channel network; and a second intrinsic channel network formed in the body and configured to allow for active flow of a fluid to support viability and/or function of the cells in the first intrinsic channel network, wherein the first intrinsic channel network comprises a first channel, the second intrinsic channel network comprises a second channel, at least a segment of the first channel and a segment of the second channel are parallel to each other and spaced apart from each other at a first distance, and the body between the segment of the first channel and the segment of the second channel forms a first barrier that allows for fluidic communication between the first channel and the second channel to nourish the cells.
Implementation 26. An implantable device comprising: a body; a first intrinsic channel network formed in the body and configured to receive cells; a second intrinsic channel network formed in the body and configured to allow for active flow of a fluid to support viability and/or function of the cells in the first intrinsic channel network, wherein the first intrinsic channel network comprises a first channel, the second intrinsic channel network comprises a second channel, at least a segment of the first channel and a segment of the second channel are parallel to each other and spaced apart from each other at a first distance, and the body between the segment of the first channel and the segment of the second channel forms a first barrier that allows for fluidic communication between the first channel and the second channel to nourish the cells; and a plurality of ports formed at a boundary of the body and in fluidic communication with the first or second intrinsic channel network, wherein each respective port in the plurality of ports is configured to couple with a corresponding tube in a plurality of tubes, thereby allowing the implantable device to anastomose with a pump or host vasculature upon implantation.
Implementation 27. An implantable device comprising: a body; a first intrinsic channel network formed in the body and configured to receive cells; a second intrinsic channel network formed in the body and configured to allow for active flow of a fluid to support viability and/or function of the cells in the first intrinsic channel network, wherein the first intrinsic channel network comprises one or more first fractal architectures, each comprising one or more generations of first channel T-junctions, the second intrinsic channel network comprises one or more second fractal architectures, each comprising one or more generations of second channel T-junctions, each respective first fractal architecture in the one or more first fractal architectures is adjacent to a corresponding second fractal architecture in the one or more second fractal architectures, thereby collectively forming a corresponding channel layer in one or more channel layers, at least a segment of each respective first fractal architecture in the one or more first fractal architectures and a segment of the corresponding second fractal architecture in the one or more second fractal architectures are parallel to each other and spaced apart from each other at a corresponding distance, and the body between the segment of each respective first fractal architecture in the one or more first fractal architectures and the segment of the corresponding second fractal architecture in the one or more second fractal architectures forms a corresponding barrier that allows for fluidic communication between the respective first fractal architecture and the corresponding second fractal architecture to nourish the cells.
Implementation 28. The implantable device of any preceding Implementation, wherein the implantable device is configured for direct intravascular implantation.
Implementation 29. The implantable device of any preceding Implementation, wherein the fluid comprises blood, medium, solute, water, or any combination thereof.
Implementation 30. The implantable device of any preceding Implementation, wherein the first nanoporous hydrogel barrier allows small molecules to diffuse across the first nanoporous hydrogel barrier.
Implementation 31. The implantable device of any preceding Implementation, wherein the first channel is a hepatobiliary channel and the second channel is a portal-venous channel.
Implementation 32. The implantable device of any preceding Implementation, wherein the segment of the first channel or the segment of the second channel has a substantially square cross-section.
Implementation 33. The implantable device of Implementation 32, wherein the substantially square cross-section has a width of from about 100 μm to about 1 mm, from 500 μm to about 5 mm, or from about 1 mm to about 10 mm.
Implementation 34. The implantable device of Implementation 33, wherein the width is about 1 mm.
Implementation 35. The implantable device of any preceding Implementation, wherein the first distance between the segment of the first channel and the segment of the second channel is from about 10 μm to about 1000 μm.
Implementation 36. The implantable device of Implementation 35, wherein the first distance is about 300 μm.
Implementation 37. The implantable device of any preceding Implementation, wherein the first channel has a nominal volume from about 1 μL to about 1000 μL.
Implementation 38. The implantable device of Implementation 37, wherein the nominal volume is about 20 p L.
Implementation 39. The implantable device of any preceding Implementation, wherein the segment of the first channel or the segment of the second channel has a length of from about 1 mm to about 100 mm.
Implementation 40. The implantable device of Implementation 39, wherein the length is about 12 mm.
Implementation 41. The implantable device of any preceding Implementation, wherein the body has a width of from about 5 mm to about 100 mm.
Implementation 42. The implantable device of Implementation 41, wherein the width is about 25 mm.
Implementation 43. The method for fabricating an implantable device, the method comprising: A) obtaining a computer-aided design (CAD) file configured for the implantable device; B) printing, in accordance with the CAD file, the implantable device using a photoink material; C) sterilizing the implantable device; and D) optionally or additionally, washing the implantable device. The implantable device comprises: a body; a first intrinsic channel network formed in the body and configured to receive cells; and a second intrinsic channel network formed in the body and configured to allow for active flow of a fluid to support viability and/or function of the cells in the first intrinsic channel network, wherein (i) the first intrinsic channel network comprises a first channel, (ii) the second intrinsic channel network comprises a second channel, (iii) at least a segment of the first channel and a segment of the second channel are parallel to each other and spaced apart from each other at a first distance, and (iii) the body between the segment of the first channel and the segment of the second channel forms a first barrier that allows for fluidic communication between the first channel and the second channel to nourish the cells.
Implementation 44. The method of Implementation 43, wherein the photoink material comprises PEGDA, GelMA or ColMA.
Implementation 45. The method of any one of Implementations 43-44, wherein the photoink material comprises 20% PEGDA, 0.5 wt % lithium phenyl-2,4,6-trimethylbenzoylphosphinate (LAP), 2 mM tartrazine, and 10% 10× polybutylene succinate (PBS).
Implementation 46. The method of any one of Implementations 43-45, wherein the printing B) comprises: B1) disposing the photoink material layer-by-layer in accordance with the CAD file; and B2) polymerizing each layer using a UV light.
Implementation 47. The method of any one of Implementations 43-46, further comprising: E) suspending cells in a solution of a collagen, thereby forming a mixture comprising the cells; F) introducing the mixture into the mixture into the first intrinsic channel network of the implantable device; and G) optionally or additionally, incubating the implantable device to allow the collagen to set.
Implementation 48. The method of Implementation 47, wherein the introducing F) is conducted by pipetting.
Implementation 49. An implantable device disclosed herein.
Implementation 50. A bioreactor system for controlling a flow through an implantable device disclosed herein.
Implementation 51. The method of fabricating an implantable device disclosed herein.
Implementation 52. The method of fabricating the implantable device of any one of Implementations 1-42.
Implementation 53. The method of culturing a plurality of cells in an implantable device disclosed herein.
Implementation 54. The method of implanting an implantable device in a subject disclosed herein.

In some embodiments, the systems, methods, and devices of the present disclosure provide a tissue device, such as a liver device. In some embodiments, the tissue device is the same as or similar to a biomimetic network, biomimetic structure, biomimetic device, vascular network device, or implantable living device disclosed in any of U.S. Patent Publication No.: 2015/0366651, U.S. Patent Publication No.: 2019/0358367, U.S. Patent Publication No.: 2018/0236134, U.S. Patent Publication No.: 2021/0071145, U.S. Patent Publication No.: 2023/0380415, and U.S. Patent Publication No.: 2023/0065127, each of which is incorporated by reference in its entirety for all purposes.

For convenience in explanation and accurate definition in the appended claims, the terms “upper,” “lower,” “up,” “down,” “upwards,” “downwards,” “inner,” “outer,” “inside,” “outside,” “inwardly,” “outwardly,” “interior,” “exterior,” “front,” “rear,” “back,” “forwards,” and “backwards” are used to describe features of the exemplary embodiments with reference to the positions of such features as displayed in the Figures.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

What is claimed is:

1. An implantable device comprising:
a body comprising a hydrogel material;
a first channel network formed in the body and configured to receive cells; and
a second intrinsic channel network formed in the body and configured to allow for active flow of a fluid to support viability and/or function of the cells in the first intrinsic channel network, wherein
the first intrinsic channel network comprises a first channel,
the second intrinsic channel network comprises a second channel,
at least a segment of the first channel and a segment of the second channel are parallel to each other and spaced apart from each other at a first distance, and
the body between the segment of the first channel and the segment of the second channel forms a first nanoporous hydrogel barrier that allows for fluidic communication between the first channel and the second channel to nourish the cells.

2. The implantable device of claim 1, wherein the device is monolithic.

3. The implantable device of claim 1, wherein the hydrogel material comprises polyethylene(glycol) diacrylate (PEGDA).

4. The implantable device of claim 3, wherein the PEGDA has a molecular weight of about 700 Da.

5. The implantable device of claim 1, wherein the hydrogel material comprises 20% PEGDA, 0.5 wt % lithium phenyl-2,4,6-trimethylbenzoylphosphinate (LAP), 2 mM tartrazine, and 10% 10× polybutylene succinate (PBS).

6. The implantable device of claim 1, wherein the hydrogel material comprises GelMA or ColMA.

7. The implantable device of claim 1, wherein the cells are suspended in a collagen matrix.

8. The implantable device of claim 7, wherein the collagen matrix comprises a collagen hydrogel.

9. The implantable device of claim 1, further comprising:
a plurality of ports formed at a boundary of the body and in fluidic communication with the first or second intrinsic channel network, wherein each respective port in the plurality of ports is configured to couple with a corresponding tube in a plurality of tubes, thereby allowing the implantable device to fluidically communicate with a pump or host vasculature.

10. The implantable device of claim 9, wherein the corresponding tube is a silastic tube.

11. The implantable device of claim 9, wherein the plurality of ports is configured to maintain a maximum principal strain of the body at a threshold level.

12. The implantable device of claim 9, wherein the plurality of ports comprises:
a first port in fluidic communication with a first end of the second intrinsic channel network and configured to couple with a first tube in the plurality of tubes and receive the fluid for the second intrinsic channel network; and
a second port in fluidic communication with a second end of the second intrinsic channel network and configured to couple with a second tube in the plurality of tubes and remove the fluid from the second intrinsic channel network.

13. The implantable device of claim 12, wherein the first and second ports are configured such that a friction connection between the first port and the first tube and connection between the second port and the second tube is stable at a pressure of about 300 mmHg.

14. The implantable device of claim 9, wherein the plurality of ports further comprises a third port in fluidic communication with the first intrinsic channel network to drain a product produced by the cells.

15. The implantable device of claim 9, wherein the plurality of ports further comprises a fourth port in fluidic communication with the second intrinsic channel network to assist in flowing the fluid in the second intrinsic channel network.

16. The implantable device of claim 9, wherein the pump is peristaltic or osmotic.

17. The implantable device of claim 1, wherein:
the first intrinsic channel network comprises one or more first fractal architectures, each comprising a first set of one or more generations bifurcations comprising a first portion extending along a first axis and at least two portions extending along a second axis perpendicular to a terminus of the first axis;
the second intrinsic channel network comprises one or more second fractal architectures, each comprising a second set of one or more generations bifurcations comprising a second portion extending along the first axis and at least two portions extending along the second axis perpendicular to the terminus of the first axis; and
each respective first fractal architecture in the one or more first fractal architectures is adjacent to a corresponding second fractal architecture in the one or more second fractal architectures, thereby collectively forming a corresponding channel layer in one or more channel layers.

18. The implantable device of claim 17, wherein the one or more channel layers is in fluidic communication with a bifurcating distributor channel network and a bifurcating collector channel network, thereby connecting each of the one or more first fractal architectures and each of the one or more second fractal architectures into a single fluidic system.

19. The implantable device of claim 17, wherein the first intrinsic channel network comprises a plurality of first fractal architectures and the second intrinsic channel network comprises a plurality of second fractal architectures, thereby forming a plurality of channel layers stacked together.

20. The implantable device of claim 19, wherein first fractal architectures in the plurality of first fractal architectures are identical to each other, and second fractal architectures in the plurality of second fractal architectures are identical to each other.


SEQUENCE LISTING (28 sequences)

SEQ ID NO: 1 - DNA, 20 bp, synthetic construct
  source 1..20: mol_type = other DNA
gcaattattccccatgaacg

SEQ ID NO: 2 - DNA, 20 bp, synthetic construct
  source 1..20: mol_type = other DNA
ggcctcactaaaccatccaa

SEQ ID NO: 3 - DNA, 21 bp, synthetic construct
  source 1..21: mol_type = other DNA
gcccagcatacgaagaaaaca

SEQ ID NO: 4 - DNA, 23 bp, synthetic construct
  source 1..23: mol_type = other DNA
tctctttgtctggaagcattcct

SEQ ID NO: 5 - DNA, 20 bp, synthetic construct
  source 1..20: mol_type = other DNA
gatgccgtgaaagagaaagc

SEQ ID NO: 6 - DNA, 20 bp, synthetic construct
  source 1..20: mol_type = other DNA
cgtgacagcactccttgttg

SEQ ID NO: 7 - DNA, 20 bp, synthetic construct
  source 1..20: mol_type = other DNA
gcgccaccaagattaactgc

SEQ ID NO: 8 - DNA, 20 bp, synthetic construct
  source 1..20: mol_type = other DNA
agcttgagctcgtacctgga

SEQ ID NO: 9 - DNA, 20 bp, synthetic construct
  source 1..20: mol_type = other DNA
cctatcctccgttacctccc

SEQ ID NO: 10 - DNA, 20 bp, synthetic construct
  source 1..20: mol_type = other DNA
attgtgtcaaacccagctcc

SEQ ID NO: 11 - DNA, 21 bp, synthetic construct
  source 1..21: mol_type = other DNA
gtcacctcagggaatgctgtg

SEQ ID NO: 12 - DNA, 21 bp, synthetic construct
  source 1..21: mol_type = other DNA
gttgacaatcttctcctgagg

SEQ ID NO: 13 - DNA, 20 bp, synthetic construct
  source 1..20: mol_type = other DNA
acccaccagcagcacacttt

SEQ ID NO: 14 - DNA, 23 bp, synthetic construct
  source 1..23: mol_type = other DNA
tctcctcctgcagtttcttctgt

SEQ ID NO: 15 - DNA, 20 bp, synthetic construct
  source 1..20: mol_type = other DNA
ccatccgtcattctctctcc

SEQ ID NO: 16 - DNA, 20 bp, synthetic construct
  source 1..20: mol_type = other DNA
tcgaacatgttgccagagtc

SEQ ID NO: 17 - DNA, 20 bp, synthetic construct
  source 1..20: mol_type = other DNA
actcccaggactggtttgtc

SEQ ID NO: 18 - DNA, 20 bp, synthetic construct
  source 1..20: mol_type = other DNA
ccagatgggaaagaggacat

SEQ ID NO: 19 - DNA, 24 bp, synthetic construct
  source 1..24: mol_type = other DNA
atggacatggctgactacagtgct

SEQ ID NO: 20 - DNA, 24 bp, synthetic construct
  source 1..24: mol_type = other DNA
acagcttgaggctccgtagtgttt

SEQ ID NO: 21 - DNA, 20 bp, synthetic construct
  source 1..20: mol_type = other DNA
aatgcaatccgttttggaag

SEQ ID NO: 22 - DNA, 20 bp, synthetic construct
  source 1..20: mol_type = other DNA
gccagagatttgaggtctgc

SEQ ID NO: 23 - DNA, 22 bp, synthetic construct
  source 1..22: mol_type = other DNA
catggattgcacatttgaagac

SEQ ID NO: 24 - DNA, 22 bp, synthetic construct
  source 1..22: mol_type = other DNA
gcaggagaagagaagctctcag

SEQ ID NO: 25 - DNA, 20 bp, synthetic construct
  source 1..20: mol_type = other DNA
ggcatcagactccagcatca

SEQ ID NO: 26 - DNA, 19 bp, synthetic construct
  source 1..19: mol_type = other DNA
gcaggcccatagggatgtt

SEQ ID NO: 27 - DNA, 20 bp, synthetic construct
  source 1..20: mol_type = other DNA
gcatggattcgtctcacgtc

SEQ ID NO: 28 - DNA, 21 bp, synthetic construct
  source 1..21: mol_type = other DNA
tttggacatgctggtgaggtt